SEQUENCE LISTING (37 sequences)

SEQ ID NO: 1 - protein, 113 aa, MusMusculus
EVQLQQSGPDLVKPGASVKISCKASGYSFTGYYIHWVKQSHGKSLEWIGRVIPNNGGTSYNQKFKGKAILTVDKSSSTAYMELRSLTSEDSAVYYCAREGIYWWGHGTTLTVS

SEQ ID NO: 2 - protein, 116 aa, Artificial
  Synthetic Construct
EVQLVESGGGLVQPGGSLRLSCAASGFTFSSYAMSWVRQAPGKGLEWVAVISGDGGSTYYADSVKGRFTISRDNSKNTLYLQMNSLRAEDTAVYYCARGRGGGSDYWGQGTLVTVS

SEQ ID NO: 3 - protein, 113 aa, Artificial
  Synthetic Construct
EVQLVESGGGLVQPGGSLRLSCAASGYSFTGYYIHWVRQAPGKGLEWVARVIPNNGGTSYNQKFKGRFTISRDNSKNTLYLQMNSLRAEDTAVYYCAREGIYWWGQGTLVTVS

SEQ ID NO: 4 - protein, 113 aa, Artificial
  Synthetic Construct
EVQLVESGGGLVQPGGSLRLSCAASGYSFTGYYIHWVRQAPGKGLEWVARVIPNNGGTSYNQKFKGRFTISVDNSKNTLYLQMNSLRAEDTAVYYCAREGIYWWGQGTLVTVS

SEQ ID NO: 5 - protein, 113 aa, Artificial
  Synthetic Construct
EVQLVESGGGLVQPGGSLRLSCAASGYSFTGYYIHWVRQAPGKGLEWVARVIPNNGGTSYNQKFKGRFTISRDKSKNTLYLQMNSLRAEDTAVYYCAREGIYWWGQGTLVTVS

SEQ ID NO: 6 - protein, 113 aa, Artificial
  Synthetic Construct
EVQLVESGGGLVQPGGSLRLSCAASGYSFTGYYIHWVRQAPGKGLEWVARVIPNNGGTSYNQKFKGRATLSVDNSKNTAYLQMNSLRAEDTAVYYCAREGIYWWGQGTLVTVS

SEQ ID NO: 7 - protein, 113 aa, Artificial
  Synthetic Construct
EVQLVESGGGLVQPGGSLRLSCAASGYSFTGYYIHWVRQAPGKGLEWVARVIPNNGGTSYNQKFKGRFTLSVDNSKNTAYLQMNSLRAEDTAVYYCAREGIYWWGQGTLVTVS

SEQ ID NO: 8 - protein, 113 aa, Artificial
  Synthetic Construct
EVQLVESGGGLVQPGGSLRLSCAASGYSFTGYYIHWVRQAPGKGLEWVARVIPNNGGTSYNQKFKGRATISVDNSKNTAYLQMNSLRAEDTAVYYCAREGIYWWGQGTLVTVS

SEQ ID NO: 9 - protein, 113 aa, Artificial
  Synthetic Construct
EVQLVESGGGLVQPGGSLRLSCAASGYSFTGYYIHWVRQAPGKGLEWVARVIPNNGGTSYNQKFKGRATLSVDNSKNTLYLQMNSLRAEDTAVYYCAREGIYWWGQGTLVTVS

SEQ ID NO: 10 - protein, 113 aa, Artificial
  Synthetic Construct
EVQLVESGGGLVQPGGSLRLSCAASGYSFTGYYIHWVRQAPGKGLEWVARVIPNAGGTSYNQKFKGRFTLSVDNSKNTAYLQMNSLRAEDTAVYYCAREGIYWWGQGTLVTVS

SEQ ID NO: 11 - protein, 113 aa, Artificial
  Synthetic Construct
EVQLVESGGGLVQPGGSLRLSCAASGYSFTGYYIHWVRQAPGKGLEWVARVIPNQGGTSYNQKFKGRFTLSVDNSKNTAYLQMNSLRAEDTAVYYCAREGIYWWGQGTLVTVS

SEQ ID NO: 12 - protein, 113 aa, MusMusculus
DVVVTQTPLSLPVSLGAQASISCRSSQSLVHSNGNTFLHWYLQKPGQSPKLLIYTVSNRFSGVPDRFSGSGSGTDFTLKISRVEAEDLGVYFCSQTTHVPWTFGGGTKLEIQR

SEQ ID NO: 13 - protein, 108 aa, Artificial
  Synthetic Construct
DIQMTQSPSSLSASVGDRVTITCRASQSISNYLAWYQQKPGKAPKLLIYAASSLESGVPSRFSGSGSGTDFTLTISSLQPEDFATYYCQQYNSLPWTFGGGTKLEIQR

SEQ ID NO: 14 - protein, 113 aa, Artificial
  Synthetic Construct
DIQMTQSPSSLSASVGDRVTITCRSSQSLVHSNGNTFLHWYQQKPGKAPKLLIYTVSNRFSGVPSRFSGSGSGTDFTLTISSLQPEDFATYYCSQTTHVPWTFGQGTKVEIKR

SEQ ID NO: 15 - protein, 113 aa, Artificial
  Synthetic Construct
DVQVTQSPSSLSASVGDRVTITCRSSQSLVHSNGNTFLHWYQQKPGKAPKLLIYTVSNRFSGVPSRFSGSGSGTDFTLTISSLQPEDFATYYCSQTTHVPWTFGQGTKVEIKR

SEQ ID NO: 16 - protein, 113 aa, Artificial
  Synthetic Construct
DIQMTQSPSSLSASVGDRVTITCRSSQSLVHSNGNTFLHWYQQKPGKAPKLLIYTVSNRFSGVPSRFSGSGSGTDFTLTISSLQPEDFATYFCSQTTHVPWTFGQGTKVEIKR

SEQ ID NO: 17 - DNA, 1392 bp, Artificial
  Synthetic Construct
atgggatggtcatgtatcatcctttttctagtagcaactgcaactggagtacattcagaagttcagctggtggagtctggcggtggcctggtgcagccagggggctcactccgtttgtcctgtgcagcttctggctacagcttcaccggttattacatccactgggtccgtcaggccccgggtaagggcctggaatgggttgcaagggttattcctaacgccggcggtaccagttataaccagaagttcaagggccgtttcacattgagcgtcgacaattccaaaaacacagcatacctgcagatgaacagcctgcgtgctgaggacactgccgtctattattgtgctcgagagggtatctactggtggggtcaaggaaccctggtcaccgtctcctcggcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 18 - protein, 463 aa, Artificial
  Synthetic Construct
MGWSCIILFLVATATGVHSEVQLVESGGGLVQPGGSLRLSCAASGYSFTGYYIHWVRQAPGKGLEWVARVIPNAGGTSYNQKFKGRFTLSVDNSKNTAYLQMNSLRAEDTAVYYCAREGIYWWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 444 aa, Artificial
  Synthetic Construct
EVQLVESGGGLVQPGGSLRLSCAASGYSFTGYYIHWVRQAPGKGLEWVARVIPNAGGTSYNQKFKGRFTLSVDNSKNTAYLQMNSLRAEDTAVYYCAREGIYWWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - DNA, 717 bp, Artificial
  Synthetic Construct
atgggatggtcatgtatcatcctttttctagtagcaactgcaaccggtgtacattcagatatccagatgacccagtccccgagctccctgtccgcctctgtgggcgatagggtcaccatcacctgcagatccagtcaaagcttagtacatagcaatggtaacactttcctccactggtatcaacagaaaccaggaaaagctccgaaactactgatttacactgttagcaaccggttctctggagtcccttctcgcttctctggatccggttctgggacggatttcactctgaccatcagcagtctgcagccagaagacttcgctacgtatttctgtagtcagactactcatgttccatggacatttggacagggtaccaaggtggagatcaaacgaactgtggctgcaccatctgtcttcatcttcccgccatctgatgagcagttgaaatctggaactgcttctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaa

SEQ ID NO: 21 - protein, 238 aa, Artificial
  Synthetic Construct
MGWSCIILFLVATATGVHSDIQMTQSPSSLSASVGDRVTITCRSSQSLVHSNGNTFLHWYQQKPGKAPKLLIYTVSNRFSGVPSRFSGSGSGTDFTLTISSLQPEDFATYFCSQTTHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 219 aa, Artificial
  Synthetic Construct
DIQMTQSPSSLSASVGDRVTITCRSSQSLVHSNGNTFLHWYQQKPGKAPKLLIYTVSNRFSGVPSRFSGSGSGTDFTLTISSLQPEDFATYFCSQTTHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - DNA, 336 bp, Mus musculus
  CDS (1)..(336)
gatgttgtggtgacccaaactccactctccctgcctgtcagtcttggagctcaagcctccatctcttgcagatctagtcagagccttgtacacagtaatggaaacacctttttacattggtacctgcagaagccaggccagtctccaaaactcctgatctacacagtttccaaccgattttctggggtcccagacaggttcagtggcagtggatcagggacagatttcacactcaagatcagcagagtggaggctgaggatctgggagtttatttctgctctcaaactacacatgttccgtggacgttcggtggaggcaccaagctggaaatccaa

SEQ ID NO: 24 - protein, 112 aa, Mus musculus
DVVVTQTPLSLPVSLGAQASISCRSSQSLVHSNGNTFLHWYLQKPGQSPKLLIYTVSNRFSGVPDRFSGSGSGTDFTLKISRVEAEDLGVYFCSQTTHVPWTFGGGTKLEIQ

SEQ ID NO: 25 - protein, 16 aa, Mus musculus
RSSQSLVHSNGNTFLH

SEQ ID NO: 26 - protein, 7 aa, Mus musculus
TVSNRFS

SEQ ID NO: 27 - protein, 9 aa, Mus musculus
SQTTHVPWT

SEQ ID NO: 28 - DNA, 342 bp, Mus musculus
  CDS (1)..(342)
gaggtccagctgcagcagtctggacctgacctggtgaagcctggggcttcagtgaagatctcctgcaaggcttctggttactcattcactggctactacatacactgggtgaagcagagccatggaaagagccttgagtggattggacgtgttattcctaacaatggaggcactagttacaaccagaagttcaagggcaaggccatattaactgtagacaagtcatccagcacagcctacatggaactccgcagcctgacatctgaggactctgcggtctattactgtgcaagagaagggatctactggtggggccacggcaccactctcacagtctcctca

SEQ ID NO: 29 - protein, 114 aa, Mus musculus
EVQLQQSGPDLVKPGASVKISCKASGYSFTGYYIHWVKQSHGKSLEWIGRVIPNNGGTSYNQKFKGKAILTVDKSSSTAYMELRSLTSEDSAVYYCAREGIYWWGHGTTLTVSS

SEQ ID NO: 30 - protein, 6 aa, Mus musculus
TGYYIH

SEQ ID NO: 31 - protein, 17 aa, Mus musculus
RVIPNNGGTSYNQKFKG

SEQ ID NO: 32 - protein, 4 aa, Mus musculus
EGIY

SEQ ID NO: 33 - DNA, 48 bp, Mus musculus
agatctagtcagagccttgtacacagtaatggaaacacctttttacat

SEQ ID NO: 34 - DNA, 21 bp, Mus musculus
acagtttccaaccgattttct

SEQ ID NO: 35 - DNA, 18 bp, Mus musculus
actggctactacatacac

SEQ ID NO: 36 - DNA, 51 bp, Mus musculus
cgtgttattcctaacaatggaggcactagttacaaccagaagttcaagggc

SEQ ID NO: 37 - DNA, 12 bp, Mus musculus
gaagggatctac

(19) United States
(12) Patent Application Publication
(10) Pub. No.: US 2009/0304687 A1
(43) Pub. Date: Dec. 10, 2009

(54) METHODS OF USING CD40 BINDING AGENTS

(75) Inventors: Jonathan Drachman, Bothell, WA (US); Che-leung Law, Bothell, WA (US); Tim Lewis, Bothell, WA (US)

Correspondence Address: Seattle Genetics/Fenwick, Silicon Valley Center, 801 California Street, Mountain View, CA, 94041, US

(73) Assignee: Seattle Genetics , Inc., Bothell, WA (US)

(21) Appl. No.: 12/302,743

(22) PCT Filed: Dec. 11, 2006
(86) PCT No.: PCT/US2006/047308

§ 371 Date: Mar. 18, 2009

Related U.S. Application Data
(60) Provisional application No. 60/749,246, filed on Dec. 9, 2005.
(60) Provisional application No. 60/811,353, filed on Jun. 5, 2006.
(60) Provisional application No. 60/811,301, filed on Jun. 5, 2006.
(60) Provisional application No. 60/847,234, filed on Sep. 25, 2006.

(51) Int. Cl. A61K 39/395; A61P 35/00

(52) U.S. Cl. 424/133.1

(57) ABSTRACT

Provided are methods of using CD40 binding agents for treating a CD40-associated disease.

### CONTINUITY

This application claims the benefit of U.S. Provisional Application No. 60/749,246, filed Dec. 9, 2005; U.S. Provisional Application No. 60/811,301, filed Jun. 5, 2006; U.S. Provisional Application No. 60/811,353, filed Jun. 5, 2006; and U.S. Provisional Application No. 60/847,234, filed Sep. 25, 2006; each of which is incorporated by reference herein in its entirety.

### BACKGROUND

This invention generally relates to therapeutic uses of CD40 antibodies. More specifically, methods of using CD40 antibodies for the treatment of various diseases or disorders characterized by cells expressing CD40 are disclosed.

CD40 is a type I integral membrane glycoprotein and a member of the tumor necrosis factor (TNF) receptor superfamily. CD40 is expressed on a variety of cell types including normal and neoplastic B cells, interdigitating cells, basal epithelial cells and carcinomas. It is also present on monocytes, macrophages, some endothelial cells, and follicular dendritic cells. CD40 is expressed early in B cell ontogeny, appearing on B cell precursors subsequent to the appearance of CD10 and CD19, but prior to expression of CD21, CD23, CD24, and appearance of surface immunoglobulin M (sIgM) (Uckun et al., 1990, Blood 15:2449). Although early reports indicated that CD40 was lost upon terminal differentiation of B cells into plasma cells, CD40 has been detected on tonsil and bone marrow-derived plasma cells (Pellat-Decounynck et al., 1994, Blood 84:2597).

The interaction of CD40 with its ligand and counter-receptor, CD40L (also referred to as CD154, gp39, and TRAP), induces both humoral and cell-mediated immune responses. CD40L is a transmembrane protein expressed predominantly on activated lymphocytes. CD4⁺ T cells. Like other proteins in the TNF family, the structure of CD40L is that of a noncovalent trimer. CD40-mediated signaling appears to be required for B cell proliferation, immunoglobulin (Ig) isotype switching, germinal center formulation, and memory B cell commitment in response to T cell-dependent antigen. CD40 binding of CD40L results in CD40 multimerization, the generation of activation signals for antigen presenting cells such as dendritic cells, monocytes, and B cells, and the generation of growth and differentiation signals for cytokine-activated fibroblasts and epithelial cells. CD40 signals are reported to be transduced from the multimerized receptor via a variety of pathways, including recruitment of a series of TNF receptor associated factors (“TRAFs”) (Kehry, 1996, J. Immumol. 156:2345-2348). Subsets of TRAFs interact differentially with TNF receptor family members, including CD40, providing stimuli to a wide variety of downstream pathways. TRAF1 and TRAF2 are implicated in the modulation of apoptosis (Speiser et al., 1997, J. Exp. Med. 185:1777-1783; Yeh et al., 1997, Immunity 7:715-725). TRAFs 2, 5, and 6 participate in proliferation and activation events. In normal B cells, binding of CD40 to CD40L recruits TRAF2 and TRAF3 to the receptor complex and induces down regulation of other TRAF's (Kuhne et al., 1997, J. Exp. Med. 186:337-342).

Apoptosis and CD40-mediated signaling are closely linked during B cell development and differentiation. A primary function of apoptosis in B cells is the clonal deletion of immature B cells, which is thought to result from extensive cross-linking of surface Ig in immature B cells. The fate of mature B cells is also modulated by a combination of signaling via surface Ig and signals derived form activated T cells, presumably mediated by CD40L molecules. A combination of signals from surface Ig and CD40 can override the apoptotic pathway and maintain germinal center B cell survival. This rescue from apoptosis in germinal centers is critical for the development of affinity antibody-producing memory B cells.

In both T and B cell malignancies, antitumor effects (growth arrest with or without apoptosis) often result when malignant cells are exposed to stimuli that lead to activation of normal lymphocytes. This activation-induced growth arrest has been observed with signals through either antigen receptors or costimulatory receptors (Ashwell et al., 1987, Science 237:61; Bridges et al., 1987, J. Immumol. 139:4242; Page and Defranco, 1988 J. Immunol. 140:3717; and Beckwith et al., 1990, J. Natl. Cancer Inst. 82:501). CD40 stimulation by certain stimulatory CD40 antibodies or soluble CD40L directly inhibits B cell lymphoma growth (Funakoshi et al., 1994, Blood 83:2787-2784; Francisco et al., 2000, Cancer Res. 60:3225-31).

The effects of stimulatory antibodies differ, depending on the type of antibodies. Stimulatory CD40 antibodies can be of different types, such as: (1) those that deliver a stimulatory signal through CD40 but do not increase the interaction between CD40 and CD40L, e.g., G28-5, (Ledbetter et al., U.S. Pat. No. 5,182,368; PCT Publication WO 96/18413), or decrease the interaction between CD40 and CD40L; and (2) those that deliver a stimulatory signal through CD40 and can increase the interaction between CD40 and CD40L, e.g., S2C6 (Francisco et al., 2000, Cancer Res. 60:3225-31). Administration of the first type of antibody has been reported to be associated with an undesirable cytokine release. The effect of the administration of the second type of antibody has not previously been reported.

### BRIEF SUMMARY

The present invention provides methods of using CD40 binding agents for the treatment of diseases and disorders characterized by cells expressing the CD40 surface antigen. The CD40 binding agents can deliver a stimulatory signal to human B cells, enhance the interaction between CD40 and CD40L, and have in vivo anti-neoplastic activity. The CD40 binding agents can be used in the treatment of a variety of diseases or disorders characterized by the proliferation of cells expressing the CD40 surface antigen.

In some aspects, the CD40 binding agent increases the binding of CD40 ligand to CD40 by at least 45%, by at least 50%, by at least 60% or by at least 75%. In various embodiments, the CD40 binding agent can, for example, block proliferation or otherwise arrest the growth of a cancer cell or cause its depletion, death, or otherwise its deletion, for example, through binding the CD40 surface antigen.

The CD40 binding agents each include at least a portion that specifically recognizes an epitope CD40, typically human CD40. In some embodiments the CD40 binding agent includes at least an antigen-binding fragment of an antibody that specifically binds to CD40.

In some embodiments, methods are provided for the treatment or prevention of a CD40-associated disorder. The methods generally include administering to a patient in need thereof an initial dose of a CD40 binding agent which (i) immunospecifically binds to CD40; and (ii) increases the binding of CD40 ligand to cell surface CD40 on B cells by at least 45% (b). A second dose of the CD40 binding agent is also administered to the patient. The initial dose is typically less than the second dose, whereby the patient exhibits reduced cytokine release, as compared to the second dosage of the agent, if administered alone. The initial dosage can be, for example, from about 0.5 mg/kg to about 8 mg/kg.

In some embodiments, the CD40 binding agent is a humanized, chimeric or human antibody that specifically binds to CD40, or an antigen binding fragment thereof. For example, the antibody or antigen-binding fragment can include a heavy chain variable domain and/or light chain variable region domain. The heavy chain variable region domain can include a framework region having an amino acid sequence at least 90% identical to the amino acid sequence of the human variable domain heavy chain subgroup III consensus amino acid sequence of SEQ ID NO:2, and at least one CDR having an amino acid sequence at least 90% identical to a corresponding heavy chain CDR of SEQ ID NO:3. The light chain variable domain can include a framework region having an amino acid sequence at least 90% identical to the human variable domain light chain subgroup kappa I consensus amino acid sequence of SEQ ID NO:13, and at least one CDR having an amino acid sequence at least 90% identical to a corresponding light chain CDR of SEQ ID NO:14.

In some embodiments, each heavy chain CDR is at least 90% identical to the corresponding heavy chain CDR of SEQ ID NO:3. In some embodiments, the heavy chain CDRs include the amino acid sequences of the heavy chain CDR1, CDR2 and CDR3 of SEQ ID NO:3. In some embodiments, each light chain CDR is at least 90% identical to the corresponding light chain CDR of SEQ ID NO:14. In some embodiments, the light chain CDRs include the amino acid sequences of the CDR1, CDR2 and CDR3 of SEQ ID NO:14.

In some embodiments, the antibody or antigen-binding fragment includes a heavy chain variable domain having the amino acid sequence of SEQ ID NO:3, SEQ ID NO:4, SEQ ID NO:5, SEQ ID NO:6, SEQ ID NO:7, SEQ ID NO:8, SEQ ID NO:9, SEQ ID NO:10, or SEQ ID NO:11. In some embodiments, the antibody or antigen-binding fragment includes a light chain variable domain having the amino acid sequence of SEQ ID NO:14, SEQ ID NO:15, or SEQ ID NO:16. In some embodiments, the antibody or antigen-binding fragment has the heavy chain variable domain amino acid sequence of SEQ ID NO:3, SEQ ID NO:4, SEQ ID NO:5, SEQ ID NO:6, SEQ ID NO:7, SEQ ID NO:8, SEQ ID NO:9, SEQ ID NO:10, or SEQ ID NO:11, and the light chain variable domain amino acid sequence of SEQ ID NO:14, SEQ ID NO:15, or SEQ ID NO:16.

In some embodiments, the heavy chain variable domain and the light chain variable domain include the amino acid sequences of SEQ ID NO:3 and SEQ ID NO:14, respectively; SEQ ID NO:4 and SEQ ID NO:14, respectively; SEQ ID NO:5 and SEQ ID NO:14, respectively; SEQ ID NO:6 and SEQ ID NO:14, respectively; SEQ ID NO:7 and SEQ ID NO:14, respectively; SEQ ID NO:8 and SEQ ID NO:14; respectively; SEQ ID NO:9 and SEQ ID NO:14, respectively; SEQ ID NO:6 and SEQ ID NO:15, respectively; SEQ ID NO:6 and SEQ ID NO:16, respectively; SEQ ID NO:7 and SEQ ID NO:16, respectively; SEQ ID NO:10 and SEQ ID NO:14, respectively; SEQ ID NO:11 and SEQ ID NO:14, respectively; SEQ ID NO:10 and SEQ ID NO:16, respectively; or SEQ ID NO:11 and SEQ ID NO:16, respectively.

The CD40 binding agent can include a human IgG constant region, such as, for example, an IgG constant region of isotype IgG1, IgG2, IgG3, or IgG4. The binding agent can include a light chain constant domain, such as, for example, a kappa constant domain.

In some embodiments, the binding agent is an antibody such as hu sgn-0, hu sgn-1, hu sgn-2, hu sgn-4, hu sgn-14, hu sgn-15, hu sgn-16, hu sgn-17, hu sgn-18, hu sgn-19, hu sgn-22, hu sgn-23, hu sgn-26 or hu sgn-27. In some embodiments, the CD40 binding agent competes for binding with monoclonal antibody S2C6 that is secreted by a hybridoma having ATCC Accession No. PTA-110.

The CD40 binding agent can be an antigen-binding fragment of an antibody, such as a Fab, a Fab′, a F(ab′)2, a Fv fragment, a diabody, a single-chain antibody, an scFv fragment or an scFv-Fc. The CD40 binding agent can optionally be labeled or conjugated to a chemotherapeutic agent, such as an auristatin (e.g., MMAE or MMAF).

Also provided is a kit including a CD40 binding agent in a container. The kit can optionally include an additional component(s), such as instructions for using the antibody to treat or prevent a CD40-associated disease.

Pharmaceutical compositions comprising a CD40 binding agent and a pharmaceutically acceptable excipients(s) are also provided.

In some embodiments, isolated polynucleotides encoding a humanized heavy chain variable region and/or a humanized light chain variable region are provided. A polynucleotide can, for example, encode the heavy chain variable domain amino acid sequence of SEQ ID NO:3, SEQ ID NO:4, SEQ ID NO:5, SEQ ID NO:6, SEQ ID NO:7, SEQ ID NO:8, SEQ ID NO:9, SEQ ID NO:10, or SEQ ID NO:11. Apolynucleotide also can, for example, encode the light chain variable domain amino acid sequence of SEQ ID NO:14, SEQ ID NO:15, or SEQ ID NO:16.

In some embodiments, isolated polynucleotide encodes the heavy chain variable domain amino acid sequence and the light chain variable domain amino acid sequence of SEQ ID NO:3 and SEQ ID NO:14, respectively; SEQ ID NO:4 and SEQ ID NO:14, respectively; SEQ ID NO:5 and SEQ ID NO:14, respectively; SEQ ID NO:6 and SEQ ID NO:14, respectively; SEQ ID NO:7 and SEQ ID NO:14, respectively; SEQ ID NO:8 and SEQ ID NO:14, respectively; SEQ ID NO:9 and SEQ ID NO:14, respectively; SEQ ID NO:6 and SEQ ID NO:15, respectively; SEQ ID NO:6 and SEQ ID NO:16, respectively; SEQ ID NO:7 and SEQ ID NO:16, respectively; SEQ ID NO:10 and SEQ ID NO:14, respectively; SEQ ID NO:11 and SEQ ID NO:14, respectively; SEQ ID NO:10 and SEQ ID NO:16, respectively; or SEQ ID NO:11 and SEQ ID NO:16, respectively.

In some embodiments, methods for inhibiting the growth of cells expressing human CD40 antigen are provided. The methods include administering a CD40 binding agent to the cells, which CD40 binding agent binds to the human cell surface CD40 antigen. The binding of the agent to the CD40 antigen inhibits the growth or differentiation of the cells.

In some embodiments, methods for treating a patient having a CD40-associated disorder are provided. The methods include administering to the patient a CD40 binding agent, which binding agent binds to human CD40. The binding of the CD40 binding agent to CD40 inhibits the growth or differentiation of cells of the CD40-associated disorder. The CD40-associated disorder can be, for example, chronic lymphocytic leukemia, Burkitt's lymphoma, multiple myeloma, a T cell lymphoma, non-Hodgkin's Lymphoma, Hodgkin's Disease, Waldenstrom's macroglobulinemia or Kaposi's sarcoma.

In some embodiments, methods for inducing depletion of peripheral B cells are provided. The methods include administering to the cells a CD40 binding agent, which binding fragment binds to a human cell surface CD40 antigen. The binding of the agent to the CD40 antigen induces depletion of the cells. The peripheral B cells can, for example, exhibit autoimmune reactivity in a patient.

The invention will best be understood by reference to the following detailed description including the preferred embodiments, taken in conjunction with the accompanying drawings and sequence listing. The discussion below is descriptive, illustrative and exemplary and is not to be taken as limiting the scope defined by any of the appended claims.

### BRIEF DESCRIPTION OF THE DRAWINGS

FIGS. 1A and 1B show the polypeptide (SEQ ID NO:18) and the coding (SEQ ID NO:17) and complementary DNA sequences of the heavy chain of a humanized CD40 antibody. The polypeptide sequence is annotated to indicate the position of the leader sequence, the variable region, and the human IgG₁ constant region. FIG. 1C shows the polypeptide (SEQ ID NO:21) and the coding (SEQ ID NO:20) and complementary DNA sequences of the light chain of a humanized CD40 antibody. The polypeptide sequence is annotated to indicate the position of the leader sequence, the variable region, and the human kappa constant region.

FIG. 2 the effect of humanized CD40 antibody on the signaling pathway in NHL (Ramos cells).

FIG. 3 shows a humanized CD40 antibody upregulates Bid protein and promotes apoptosis in vitro.

FIG. 4 shows the effect of treatment with a control antibody, a murine anti-CD40 antibody, and a humanized anti-CD40 antibody on tumor volume measured over a two-week period, with treatment beginning 13 days post-tumor tumor transplant.

FIG. 5 shows the effect of treatment with a control antibody, a murine anti-CD40 antibody, and a humanized anti-CD40 antibody, on survival of tumor-bearing mice.

FIG. 6A shows the results in a lymphoma model of treatment with a CD40 antibody alone or in combination with CHOP.

FIG. 6B shows the results in a lymphoma model of treatment with a CD40 antibody alone or in combination with CHOP or prednisone.

FIG. 7 shows a comparison of SGN-40 and Rituximab in an NHL Xenograft model.

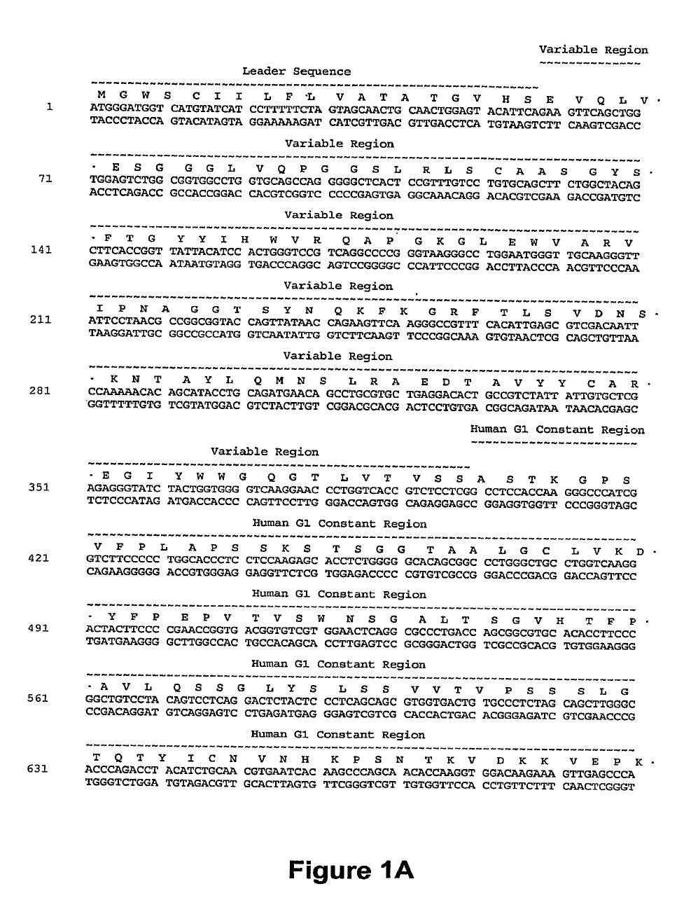

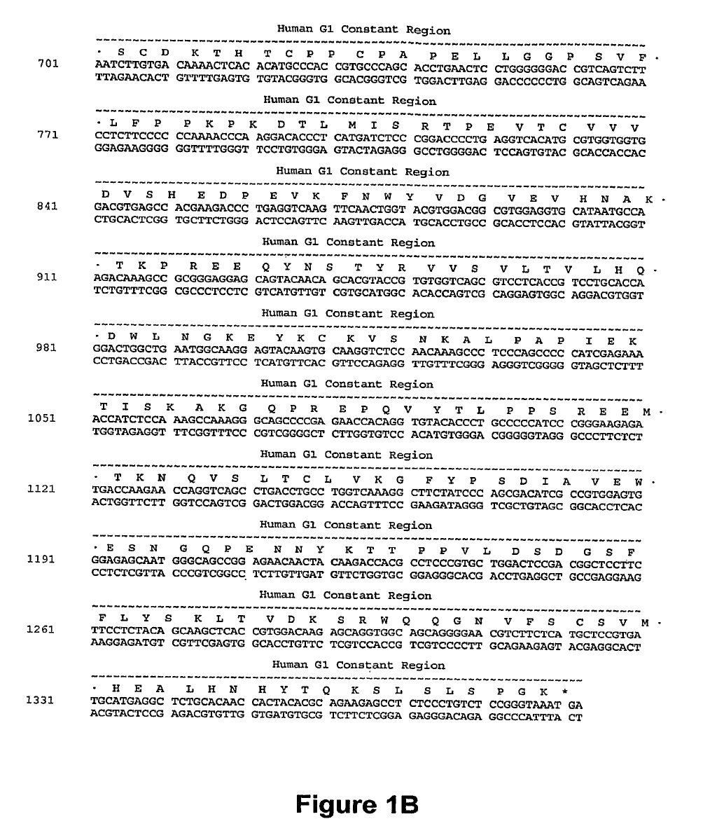

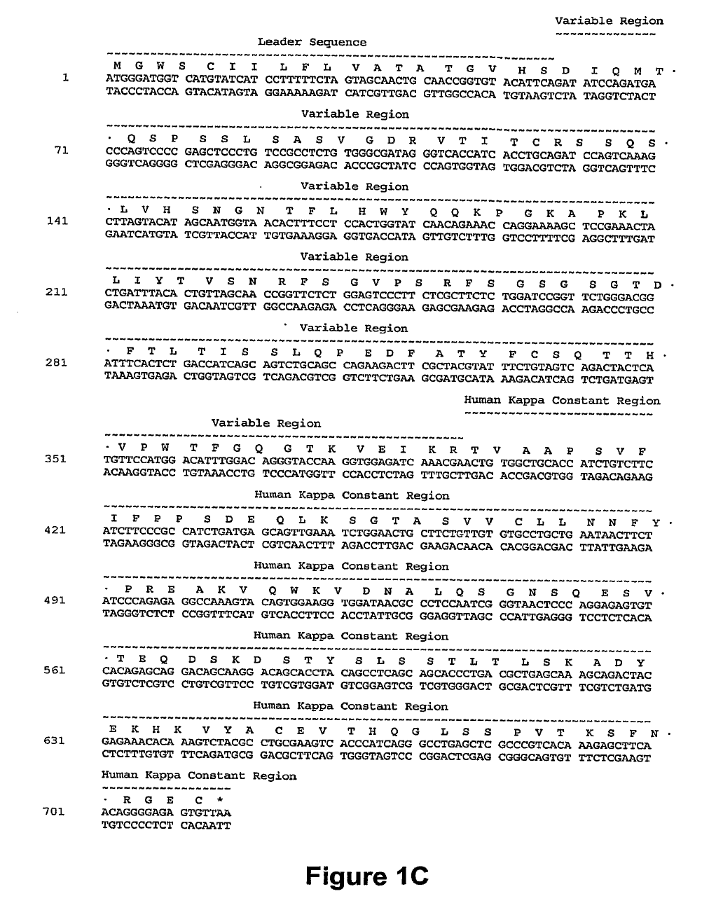

### DETAILED DESCRIPTION

For clarity of disclosure, and not by way of limitation, the detailed description of the invention is divided into the subsections which follow.

When trade names are used herein, the trade name also refers to the trade name product formulation, the generic drug, and the active pharmaceutical ingredient(s) of the trade name product, unless otherwise indicated by context.

Unless defined otherwise, all technical and scientific terms used herein have the same meaning as commonly understood by one of ordinary skill in the art pertinent to the methods and compositions described.

### DEFINITIONS

The terms “CD40” and “CD40 surface antigen” refer to a 50 kD glycoprotein expressed on the surface of normal and neoplastic B cells, which acts as a receptor for signals involved in cellular proliferation and differentiation and is sometimes referred to as Bp50 (Ledbetter et al., 1987, J. Immunol. 138:788-785). A cDNA molecule encoding CD40 has been isolated from a library prepared from the Burkitt's lymphoma cell line Raji (Stamenkovic et al., 1989, EMBO J. 8:1403). A cell that expresses CD40 is any cell characterized by the surface expression of CD40, including, but not limited to, normal and neoplastic B cells, interdigitating cells, basal epithelial cells, carcinoma cells, macrophages, endothelial cells, follicular dendritic cells, tonsil cells, and bone marrow-derived plasma cells. In some embodiments, the CD40 molecule is a human CD40 molecule.

The terms, “CD40 antigen epitope” and “CD40 epitope”, as used herein, refer to a molecule (e.g., a peptide) or a fragment of a molecule capable of immunoreactivity with a CD40 antibody and, for example, includes a CD40 antigenic determinant recognized by the S2C6 monoclonal antibody. CD40 antigen epitopes can be included in proteins, protein fragments, peptides or the like. The epitopes are most commonly proteins, short oligopeptides, oligopeptide mimics (i.e., organic compounds that mimic antibody binding properties of the CD40 antigen), or combinations thereof.

As used herein, “specific binding” and “specifically binds” refer to binding to a predetermined antigen by a CD40 binding agent. Typically, the agent binds with an affinity of at least about 1×10⁷ M⁻¹, and binds to the predetermined antigen with an affinity that is at least two-fold greater than its affinity for binding to a non-specific antigen (e.g., BSA, casein) other than the predetermined antigen or a closely-related antigen.

“Native antibodies” and “native immunoglobulins” are defined herein as heterotetrameric glycoproteins, typically of about 150,000 daltons, composed of two identical light (L) chain and two identical heavy (H) chains. Each light chain is covalently linked to a heavy chain by one disulfide bond to form a heterodimer. The heterotetramer is formed by covalent disulfide linkage between the two identical heavy chains of such heterodimers. Although the light and heavy chains are linked together by one disulfide bond, the number of disulfide linkages between the two heavy chains varies by immunoglobulin isotype. Each heavy and light chain also has regularly spaced intrachain disulfide bridges. Each heavy chain has at the amino-terminus a variable domain (V_{H}), followed by three or four constant domains (C_{H}1, C_{H}2, C_{H}3, and C_{H}4), as well as a hinge region between C_{H}1 and C_{H}2. Each light chain has two domains, an amino-terminal variable domain (V_{L}) and a carboxy-terminal constant domain (C_{L}). The V_{L} domain associates non-covalently with the V_{H} domain, whereas the C_{L} domain is commonly covalently linked to the C_{H}1 domain via a disulfide bond. Particular amino acid residues are believed to form an interface between the light and heavy chain variable domains (Chothia et al., 1985, J. Mol. Biol. 186:651-663.)

The term “hypervariable” refers to the fact that certain sequences within the variable domains differ extensively in sequence among antibodies and contain residues that are directly involved in the binding and specificity of each particular antibody for its specific antigenic determinant. Hypervariability, both in the light chain and the heavy chain variable domains, is concentrated in three segments known as complementarity determining regions (CDRs) or hypervariable loops (HVLs). CDRs are defined by sequence comparison in Kabat et al., 1991, In: Sequences of Proteins of Immunological Interest, 5^{th} Ed. Public Health Service, National Institutes of Health, Bethesda, Md., whereas HVLs are structurally defined according to the three-dimensional structure of the variable domain, as described by Chothia and Lesk, 1987, J. Mol. Biol. 196: 901-917. Where these two methods result in slightly different identifications of a CDR, the structural definition is preferred. As defined by Kabat, CDR-L1 is positioned at about residues 24-34, CDR-L2, at about residues 50-56, and CDR-L3, at about residues 89-97 in the light chain variable domain; CDR-H1 is positioned at about residues 31-35, CDR-H2 at about residues 50-65, and CDR-H3 at about residues 95-102 in the heavy chain variable domain.

The three CDRs within each of the heavy and light chains are separated by framework regions (FR), which contain sequences that tend to be less variable. From the amino terminus to the carboxy terminus of the heavy and light chain variable domains, the FRs and CDRs are arranged in the order: FR1, CDR1, FR2, CDR2, FR3, CDR3, and FR4. The largely β-sheet configuration of the FRs brings the CDRs within each of the chains to close proximity to each other as well as to the CDRs from the other chain. The resulting conformation contributes to the antigen binding site (see Kabat et al., 1991, NIH Publ. No. 91-3242, Vol. I, pages 647-669), although not all CDR residues are necessarily directly involved in antigen binding.

FR residues and Ig constant domains are not directly involved in antigen binding, but contribute to antigen binding and/or mediate antibody effector function. Some FR residues can have a significant effect on antigen binding in at least three ways: by noncovalently binding directly to an epitope, by interacting with one or more CDR residues, and by affecting the interface between the heavy and light chains. The constant domains are not directly involved in antigen binding but mediate various Ig effector functions, such as participation of the antibody in antibody dependent cellular cytotoxicity (ADCC), complement dependent cytotoxicity (CDC) and antibody dependent cellular phagocytosis (ADCP).

The light chains of vertebrate immunoglobulins are assigned to one of two clearly distinct classes, kappa (κ) and lambda (λ), based on the amino acid sequence of the constant domain. By comparison, the heavy chains of mammalian immunoglobulins are assigned to one of five major classes, according to the sequence of the constant domains: IgA, IgD, IgE, IgG, and IgM. IgG and IgA are further divided into subclasses (isotypes), e.g., IgG₁, IgG₂, IgG₃, IgG₄, IgA₁, and IgA2. The heavy chain constant domains that correspond to the different classes of immunoglobulins are called α, δ, ε, γ, and μ, respectively. The subunit structures and three-dimensional configurations of the classes of native immunoglobulins are well known.

The terms, “antibody”, “CD40 antibody”, “humanized CD40 antibody”, and “variant humanized CD40 antibody” are used herein in the broadest sense and specifically encompass monoclonal antibodies (including full length monoclonal antibodies), polyclonal antibodies, multispecific antibodies (e.g., bispecific antibodies), and antibody fragments such as variable domains and other portions of antibodies that exhibit a desired biological activity, e.g., CD40 binding.

The term “monoclonal antibody” (mAb) refers to an antibody obtained from a population of substantially homogeneous antibodies; that is, the individual antibodies comprising the population are identical except for naturally occurring mutations that may be present in minor amounts. Monoclonal antibodies are highly specific, being directed against a single antigenic determinant, also referred to as an epitope. The modifier “monoclonal” is indicative of a substantially homogeneous population of antibodies directed to the identical epitope and is not to be construed as requiring production of the antibody by any particular method. Monoclonal antibodies can be made by any technique or methodology known in the art; for example, the hybridoma method first described by Köhler et al., 1975, Nature 256:495, or recombinant DNA methods known in the art (see, e.g., U.S. Pat. No. 4,816,567). In another example, monoclonal antibodies can also be isolated from phage antibody libraries, using techniques described in Clackson et al., 1991, Nature 352: 624-628, and Marks et al., 1991, J. Mol. Biol. 222: 581-597.

In contrast, the antibodies in a preparation of polyclonal antibodies are typically a heterogeneous population of immunoglobulin isotypes and/or classes and also exhibit a variety of epitope specificity.

The term “chimeric” antibody as used herein is a type of monoclonal antibody in which a portion of or the complete amino acid sequence in one or more regions or domains of the heavy and/or light chain is identical with, homologous to, or a variant of the corresponding sequence in a monoclonal antibody from another species or belonging to another immunoglobulin class or isotype, or from a consensus sequence. Chimeric antibodies include fragments of such antibodies, provided that the antibody fragment exhibits the desired biological activity of its parent antibody, for example binding to the same epitope (see, e.g., U.S. Pat. No. 4,816,567; and Morrison et al., 1984, Proc. Natl. Acad. Sci. USA 81: 6851-6855).

The terms, “antibody fragment”, “CD40 antibody fragment”, “humanized CD40 antibody fragment”, “variant humanized CD40 antibody fragment” refer to a portion of a full length CD40 antibody, in which a variable region or a functional capability is retained, for example, specific CD40 epitope binding. Examples of antibody fragments include, but are not limited to, a Fab, Fab′, F(ab′)₂, Fd, Fv, scFv and scFv-Fc fragment, a diabody, a linear antibody, a single-chain antibody, a minibody, a diabody formed from antibody fragments, and multispecific antibodies formed from antibody fragments.

Certain types of antibody fragments can be generated by enzymatic treatment of a full-length antibody. Papain digestion of antibodies produces two identical antigen-binding fragments called “Fab” fragments, each with a single antigen-binding site, and a residual “Fc” fragment, so called because of its ability to crystallize readily. The Fab fragment also contains the constant domain of the light chain and the C_{H}1 domain of the heavy chain. Pepsin treatment yields a F(ab′)₂ fragment that has two antigen-binding sites and is still capable of cross-linking antigen.

Fab′ fragments differ from Fab fragments by the presence of a few additional residues at the C-terminus of the C_{H}1 domain, including one or more cysteines from the antibody hinge region. Fab-SH is the designation herein for a Fab′ in which the cysteine residue(s) of the constant domains bear a free thiol group. F(ab′)₂ antibody fragments are pairs of Fab′ fragments linked by cysteine residues in the hinge region. Other chemical couplings of antibody fragments are also known.

“Fv” is a minimum antibody fragment that contains a complete antigen-recognition and binding site consisting of a dimer of one heavy and one light chain variable domain in tight, non-covalent association. In this configuration, the three CDRs of each variable domain interact to define an antigen-biding site on the surface of the V_{H}-V_{L} dimer. Collectively, the six CDRs confer antigen-binding specificity to the antibody.

A “single-chain Fv” or “scFv” antibody fragment is a single chain Fv variant comprising the V_{H} and V_{L} domains of an antibody, in which the domains are present in a single polypeptide chain and which is capable of recognizing and binding antigen. The scFv polypeptide optionally contains a polypeptide linker positioned between the V_{H} and V_{L} domains that enables the scFv to form a desired three-dimensional structure for antigen binding (see, e.g., Pluckthun, 1994, In The Pharmacology of Monoclonal Antibodies, Vol. 113, Rosenburg and Moore eds., Springer-Verlag, New York, pp. 269-315).

The term “diabodies” refers to small antibody fragments having two antigen-binding sites. Each fragment contains a heavy chain variable domain (V_{H}) concatenated to a light chain variable domain (V_{L}). By using a linker that is too short to allow pairing between the two domains on the same chain, the linked V_{H}-V_{L} domains are forced to pair with complementary domains of another chain, creating two antigen-binding sites. Diabodies are described more fully, for example, in EP 404,097; WO 93/11161; and Hollinger et al., 1993, Proc. Natl. Acad. Sci. USA 90: 6444-6448.

The term “linear antibodies” refers to antibodies that comprise a pair of tandem Fd segments (V_{H}-C_{H}1-V_{H}-C_{H}1) that form a pair of antigen binding regions. Linear antibodies can be bispecific or monospecific as described in, for example, Zapata et al. 1995, Protein Eng. 8(10):1057-1062.

A CD40 binding agent (e.g., a humanized antibody or a humanized antibody fragment can include an immunoglobulin amino acid sequence variant, or fragment thereof, which is capable of binding to a predetermined antigen and which, comprises one or more FRs having substantially the amino acid sequence of a human immunoglobulin and one or more CDRs having substantially the amino acid sequence of a non-human immunoglobulin. This non-human amino acid sequence is referred to herein as an “import” sequence, which is typically taken from an “import” antibody domain, particularly a variable domain. In general, a humanized antibody includes at least the CDRs or HVLs of a non-human antibody, inserted between the FRs of a human heavy or light chain variable domain. In certain aspects, a humanized CD40 antibody contains CDR and/or HVL residues or sequences derived from the murine monoclonal antibody S2C6 inserted between the FRs of human consensus sequence heavy and light chain variable domains.

In another aspect, a humanized CD40 antibody comprises substantially all of at least one, and typically two, variable domains (such as contained, for example, in Fab, Fab′, F(ab′)₂, Fabc, and Fv fragments) in which all, or substantially all, of the CDRs correspond to those of a non-human immunoglobulin and all, or substantially all, of the FRs are those of a human immunoglobulin consensus sequence. In another aspect, a humanized CD40 antibody also includes at least a portion of an immunoglobulin Fc region, typically that of a human immunoglobulin. Ordinarily, the antibody will contain both the light chain as well as at least the variable domain of a heavy chain. The antibody also may include one or more of the C_{H}1, hinge, C_{H}2, C_{H}3, and/or C_{H}4 regions of the heavy chain, as appropriate.

A CD40 binding agent can include any class of immunoglobulins, including IgM, IgG, IgD, IgA and IgE, and any isotype, including IgG₁, IgG₂, IgG₃, IgG4, IgA₁ and IgA2. For example, the constant domain can be a complement fixing constant domain where it is desired that the binding agent exhibit cytotoxic activity, and the isotype is typically IgG₁. Where such cytotoxic activity is not desirable, the constant domain may be of another isotype, e.g., IgG₂. An alternative CD40 binding agent can comprise sequences from more than one immunoglobulin class or isotype, and selecting particular constant domains to optimize desired effector functions is within the ordinary skill in the art.

The FRs and CDRs, or HVLs, of CD40 antibody need not correspond precisely to the parental sequences. For example, one or more residues in the import CDR, or HVL, or the consensus FR sequence may be altered (e.g., mutagenized) by substitution, insertion or deletion such that the resulting amino acid residue is no longer identical to the original residue in the corresponding position in either parental sequence. Such alterations, however, typically will not be extensive. Usually, at least 75% of the antibody residues will correspond to those of the parental consensus FR and import CDR sequences, more often at least 90%, and most frequently greater than 95%, or greater than 98% or greater than 99%.

Immunoglobulin residues that affect the interface between heavy and light chain variable regions (“the V_{L}-V_{H} interface”) are those that affect the proximity or orientation of the two chains with respect to one another. Certain residues that may be involved in interchain interactions include V_{L} residues 34, 36, 38, 44, 46, 87, 89, 91, 96, and 98 and V_{H} residues 35, 37, 39, 45, 47, 91, 93, 95, 100, and 103 (utilizing the numbering system set forth in Kabat et al., Sequences of Proteins of Immunological Interest (National Institutes of Health, Bethesda, Md., 1987)). Additional residues include V_{L} residues 43 and 85, and V_{H} residues 43 and 60, as disclosed in U.S. Pat. No. 6,407,213, which is hereby incorporated by reference in its entirety. While these residues are indicated for human IgG only, they are applicable across species. Import antibody residues that are reasonably expected to be involved in interchain interactions are selected for substitution into the consensus sequence.

The terms “consensus sequence” and “consensus antibody” as used herein refer to an amino acid sequence which comprises the most frequently occurring amino acid residue at each location in all immunoglobulins of any particular class, isotype, or subunit structure, e.g., a human immunoglobulin variable domain. The consensus sequence may be based on immunoglobulins of a particular species or of many species. A “consensus” sequence, structure, or antibody is understood to encompass a consensus human sequence as described in certain embodiments, and to refer to an amino acid sequence which comprises the most frequently occurring amino acid residues at each location in all human immunoglobulins of any particular class, isotype, or subunit structure. Provided are consensus human structures and consensus structures which consider other species in addition to human. Thus, the consensus sequence contains an amino acid sequence having at each position an amino acid that is present in one or more known immunoglobulins, but which may not exactly duplicate the entire amino acid sequence of any single immunoglobulin. The variable region consensus sequence is not obtained from any naturally produced antibody or immunoglobulin. Useful consensus sequences include a human variable light chain kappa I consensus sequence (SEQ ID NO:13) and a human variable heavy chain subgroup III consensus sequence (SEQ ID NO:2), derived from the data provided in Kabat et al., 1991, Sequences of Proteins of Immunological Interest, 5th Ed. Public Health Service, National Institutes of Health, Bethesda, Md., and variants thereof. The FRs of heavy and light chain consensus sequences, and variants thereof, provide useful sequences for the preparation of humanized CD40 antibodies. See, for example, U.S. Pat. Nos. 6,037,454 and 6,054,297. In certain embodiments, the FR used to prepare the humanized antibodies were derived from consensus sequences for a human variable light chain kappa I consensus sequence and for a human variable heavy chain subgroup III consensus sequence.

As used herein, “variant”, “CD40 variant”, “humanized CD40 variant”, or “variant humanized CD40” each refers to a humanized CD40 antibody having at least a heavy chain variable CDR or HVL sequence derived from the murine monoclonal antibody S2C6 and FR sequences derived from human consensus sequences. Variants include those having one or more amino acid changes in one or both light chain or heavy chain variable domains, provided that the amino acid change does not substantially impair binding of the antibody to CD40. Humanized CD40 variants typically include amino acid substitutions that improve antibody performance by allowing improved folding of the antibody molecule.

An “isolated” CD40 binding agent is one that has been identified and separated and/or recovered from a component of its natural environment. Contaminant components of the agent's natural environment are those materials that may interfere with diagnostic or therapeutic uses of the agent, and can be enzymes, hormones, or other proteinaceous or nonproteinaceous solutes. In one aspect, the agent will be purified:
(a) to greater than 95% isolation by weight of the agent as determined by the Lowry method, and in another aspect, more than 99% isolation by weight, or
(b) to a degree of isolation sufficient to obtain at least 15 residues of N-terminal or internal amino acid sequence by use of a spinning cup sequenator, or
(c) to homogeneity by SDS-PAGE under reducing or nonreducing conditions as visualized using Coomassie blue or, preferably, silver stain.

An isolated agent includes an agent in situ within recombinant cells, since at least one component of the agent's natural environment will not be present. Ordinarily however, an isolated agent will be prepared by at least one purification step.

The term “antibody performance” refers to factors that contribute to antibody recognition of antigen or the effectiveness of an antibody in vivo. Changes in the amino acid sequence of an antibody can affect antibody properties such as folding, and can influence physical factors such as initial rate of antibody binding to antigen (vₒ), dissociation constant of the antibody from antigen (Kd), affinity constant of the antibody for the antigen, conformation of the antibody, protein stability, and half life of the antibody.

The term “epitope tagged” when used herein, refers to a CD40 binding agent fused to an “epitope tag”. An “epitope tag” is a polypeptide having a sufficient number of amino acids to provide an epitope for agent production, yet is designed such that it does not interfere with the desired activity of the CD40 binding agent. The epitope tag is usually sufficiently unique such that an agent raised against the epitope tag does not substantially cross-react with other epitopes. Suitable tag polypeptides generally contain at least 6 amino acid residues and usually contain about 8 to 50 amino acid residues, or about 9 to 30 residues. In certain embodiments, the epitope tag is a “salvage receptor binding epitope”. As used herein, the term “salvage receptor binding epitope” refers to an epitope of the Fc region of an IgG molecule (such as IgG₁, IgG₂, IgG₃, or IgG₄) that is responsible for increasing the in vivo serum half-life of the IgG molecule.

The term “cytotoxic agent” refers to a substance that inhibits or prevents the function of cells and/or causes destruction of cells. The term is intended to include radioactive isotopes (such as I¹¹³, I¹²⁵, Y⁹⁰, and Re¹⁸⁶), chemotherapeutic agents, and toxins such as enzymatically active toxins of bacterial, fungal, plant, or animal origin, and fragments thereof. Such cytotoxic agents can be coupled to an antibody, e.g., a humanized CD40 antibody, using known, standard procedures, and used, for example, to treat a patient indicated for therapy with the antibody.

A “chemotherapeutic agent” is a chemical compound useful in the treatment of cancer. Examples of chemotherapeutic agents include alkylating agents such a thiotepa and cyclosphosphamide (CYTOXAN™); alkyl sulfonates such as busulfan, improsulfan, and piposulfan; aziridines such as benzodopa, carboquone, meturedopa, and uredopa; ethylenimines and methylamelamines including altretamine, triethylenemelamine, trietylenephosphoramide, triethylenethiophosphoramide, and trimethylolomelamine; acetogenins (especially bullatacin and bullatacinone); camptothecin (including the synthetic analogue topotecan); bryostatin; callystatin; CC-1065 (including its adozelesin, carzelesin, and bizelesin synthetic analogues); cryptophycines (particularly cryptophycin 1 and cryptophycin 8); dolastatin, auristatins, (including analogues monomethyl-auristatin E and monomethyl-auristatin F); duocarmycin (including the synthetic analogues, KW-2189 and CBI-TMI); eleutherobin; pancratistatin; sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine; trofosfamide, uracil mustard; nitrosureas such as cammustine, chlorozotocin, fotemustine, lomustine, nimustine, ranimustine; antibiotics such as the enediyne antibiotics (e.g., calicheamicin, especially calichemicin gammalI and calicheamicin phil, see for example, Agnew, Chem. Intl. Ed. Engl., 33:183-186; dynemicin, including dynemicin A; bisphosphonates, such as clodronate; esperamicin; as well as neocarzinostatin chromophore and related chromoprotein enediyne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (Adriamycin™) (including morpholino-doxorubicin, cyanomorpholino-doxorubicin, 2-pyrrolino-doxorubicin, and deoxydoxorubicin), epirubucin, esorubicin, idarubicin, marcellomycin, mitomycins such as mitomycin C, mycophenolic acid, nogalamycin, olivomycins, peplomycin, potfiromycin, puromycine, quelamycin, rodorubicin, streptonigrin, streptozocin, tubercidin, ubenimex, zinostatin, zorubicin; anti-metabolites such a methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carnofur, cytarabine, dideoxyuridine, doxiffuridine, enocitabine, floxuridine; androgens such as calusterone, dromostanolone propionate, epitiostanol, mepitiostane, testolactone; anti-adranals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; democolcine; diaziquone; elfomithine; elliptinium acetate; an epothilone; etoglucid; gallium nitrate; hydroxyurea; lentinan; lonidamine; maytansinoids such as maytansine and ansamitocins; mitoguazone, mitoxantrone; mopidamol; nitracrine; pentostatin; phenamet; pirarubicin; losoxantrone; podophyllinic acid; 2-ethylhydrazide; procarbazine; PSKV; razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitabronitol; mitolactol; pipobroman; gacytosine; arabinoside (“Ara-C”); cyclophosphamide; thiotepa; taxoids, e.g., paclitaxel (TAXOL®, Bristol-Myers Squibb Oncology, Princeton, N.J.) and doxetaxel (TAXOTERE®, Rhone-Poulenc Rorer, Antony, France); chlorambucil; gemcitabine (Gemzar™); 6-thioguanine; mercaptopurine; methotrexate; platinum analogs such as cisplatin and carboplatin; vinblastine; platinum; etoposide (VP-16); ifosfamide; mitoxantrone; vincristine; vinorelbine (Navelbine™); novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids, or derivatives of any of the above. Also included in this definition are anti-hormonal agents that act to regulate or inhibit hormone action on tumors such as anti-estrogens and selective estrogen receptor modulators (SERMs), including, for example, tamoxifen (including Nolvadex™), raloxifene, droloxifene, 4-hydroxytamoxifen, trioxifene, keoxifene, LY117018, onapristone, and toremifene (Fareston™); aromatase inhibitors that inhibit the enzyme aromatase, which regulates estrogen production in the adrenal glands, such as, for example, 4(5)-imidazoles, aminoglutethimide, megestrol acetate (Megace™), exemestane, formestane, fadrozole, vorozole (Rivisor™), letrozole (Femara™), and anastrozole (Arimidex™); and anti-androgens such as flutamide, nilutamide, bicalutamide, leuprolide, and goserelin; and pharmaceutically acceptable salts, acids, or derivatives of any of the above.

The term “prodrug” as used herein refers to a precursor or derivative form of a pharmaceutically active substance that is less cytotoxic to tumor cells compared to the parent drug and is capable of being enzymatically activated or converted into the more active form. See, for example, Wilman, 1986, “Prodrugs in Cancer Chemotherapy”, In Biochemical Society Transactions, 14, pp. 375-382, 615th Meeting Belfast and Stella et al., 1985, “Prodrugs: A Chemical Approach to Targeted Drug Delivery, In: “Directed Drug Delivery, Borchardt et al., (ed.), pp. 247-267, Humana Press. Useful prodrugs include, but are not limited to, phosphate-containing prodrugs, thiophosphate-containing prodrugs, sulfate-containing prodrugs peptide-containing prodrugs, D-amino acid-modified prodrugs, glycosylated prodrugs, β-lactam-containing prodrugs, optionally substituted phenoxyacetamide-containing prodrugs, and optionally substituted phenylacetamide-containing prodrugs, 5-fluorocytosine and other 5-fluorouridine prodrugs that can be converted into the more active cytotoxic free drug. Examples of cytotoxic drugs that can be derivatized into a prodrug form include, but are not limited to, those chemotherapeutic agents described above.

The term “label” refers to a detectable compound or composition that is conjugated directly or indirectly to the antibody. The label may itself be detectable (e.g., radioisotope labels or fluorescent labels) or, in the case of an enzymatic label, may catalyze chemical alteration of a substrate compound or composition that is detectable. Labeled CD40 binding agents can be prepared and used in various applications including in vitro and in vivo diagnostics.

A “liposome” is a small vesicle composed of various types of lipids, phospholipids, and/or surfactant. Liposomes are useful for delivery to a mammal of a compound or formulation, such as a CD40 binding agent (e.g., humanized CD40 antibody), optionally, coupled to or in combination with one or more pharmaceutically active agents. The components of the liposome are commonly arranged in a bilayer formation, similar to the lipid arrangement of biological membranes.

An “isolated” nucleic acid molecule is a nucleic acid molecule that is identified and separated from at least one contaminant nucleic acid molecule with which it is ordinarily associated in the natural source of the nucleic acid. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes a nucleic acid molecule contained in cells that ordinarily express the encoded product where, for example, the nucleic acid molecule is in a chromosomal location different from that of natural cells.

The term “control sequences” refers to polynucleotide sequences necessary for expression of an operably linked coding sequence in a particular host organism. The control sequences suitable for use in prokaryotic cells include, for example, promoter, operator, and ribosome binding site sequences. Eukaryotic control sequences include, but are not limited to, promoters, polyadenylation signals, and enhancers. These control sequences can be utilized for expression and production of CD40 binding agents in prokaryotic and eukaryotic host cells.

A nucleic acid sequence is “operably linked” when it is placed into a functional relationship with another nucleic acid sequence. For example, a nucleic acid presequence or secretory leader is operably linked to a nucleic acid encoding a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, enhancers are optionally contiguous. Linking can be accomplished by ligation at convenient restriction sites. If such sites do not exist, synthetic oligonucleotide adaptors or linkers can be used.

As used herein, the expressions “cell”, “cell line”, and “cell culture” are used interchangeably and all such designations include the progeny thereof. Thus, “transformants” and “transformed cells” include the primary patient cell and cultures derived therefrom without regard for the number of transfers. It is also understood that all progeny may not be precisely identical in DNA content, due to deliberate or naturally occurring mutations. Mutant progeny that have the same function or biological activity as screened for in the originally transformed cell are included. Where distinct designations are intended, it will be clear from the context.

The term “mammal” for purposes of treatment refers to any animal classified as a mammal, including humans, domesticated and farm animals, and zoo, sports, or pet animals, such as dogs, horses, cats, cows, and the like. Preferably, the mammal is human.

A “disorder”, as used herein, is any condition that would benefit from treatment with a CD40 binding agent described herein. This includes chronic and acute disorders or diseases including those pathological conditions that predispose the mammal to the disorder in question. Non-limiting examples or disorders to be treated herein include cancer, hematological malignancies, benign and malignant tumors, leukemias and lymphoid malignancies and inflammatory, angiogenic and immunologic disorders.

The terms “cancer” and “cancerous” refer to or describe the physiological condition in mammals that is typically characterized by unregulated cell growth. Examples of cancer include, but are not limited to, carcinoma, lymphoma, blastoma, sarcoma, and leukemia.

As used herein, the term “CD40-associated disorder” or “CD40-associated disease” refers to a condition in which modification or elimination of cells expressing CD40 is indicated. These include CD40-expressing cells demonstrating abnormal proliferation or CD40-expressing cells that are associated with cancerous or malignant growth. More particular examples of cancers that demonstrate abnormal expression of CD40 antigen include B lymphoblastoid cells, Burkitt's lymphoma, multiple mycloma, T cell lymphomas, Kaposi's sarcoma, osteosarcoma, epidermal and endothelial tumors, pancreatic, lung, breast, ovarian, colon, prostate, head and neck, skin (melanoma), bladder, and kidney cancers. Such disorders include, but are not limited to, leukemias, lymphomas, including B cell lymphoma and non-Hodgkin's lymphoma, multiple myeloma, Waldenstrom's macroglobulinemia; solid tumors, including sarcomas, such as osteosarcoma, Ewing's sarcoma, malignant melanoma, adenocarcinoma, including ovarian adenocarcinoma, Kaposi's sarcoma/Kaposi's tumor and squamous cell carcinoma.

A CD40-associated disorder also includes diseases and disorders of the immune system, such as auto-immune disorders and inflammatory disorders. Such conditions include, but are not limited to, rheumatoid arthritis (RA), systemic lupus erythematosus (SLE), scleroderma, Sjogren's syndrome, multiple sclerosis, inflammatory bowel disease (e.g., ulcerative colitis and Crohn's disease), pulmonary inflammation, asthma, and idiopathic thrombocytopenic purara (ITP).

The phrase “arrests the growth of” or “growth inhibitory” when used herein refers to inhibiting growth or proliferation of a cell, especially a neoplastic cell type expressing the CD40 antigen. Thus, growth inhibition, for example, significantly reduces the percentage of neoplastic cells in S phase.

The term “intravenous infusion” refers to introduction of an agent into the vein of an animal or human patient over a period of time greater than approximately 15 minutes, generally between approximately 30 to 90 minutes.

The term “intravenous bolus” or “intravenous push” refers to drug administration into a vein of an animal or human such that the body receives the drug in approximately 15 minutes or less, generally 5 minutes or less.

The term “subcutaneous administration” refers to introduction of an agent under the skin of an animal or human patient, preferable within a pocket between the skin and underlying tissue, by relatively slow, sustained delivery from a drug receptacle. Pinching or drawing the skin up and away from underlying tissue may create the pocket.

The term “subcutaneous infusion” refers to introduction of a drug under the skin of an animal or human patient, preferably within a pocket between the skin and underlying tissue, by relatively slow, sustained delivery from a drug receptacle for a period of time including, but not limited to, 30 minutes or less, or 90 minutes or less. Optionally, the infusion may be made by subcutaneous implantation of a drug delivery pump implanted under the skin of the animal or human patient, wherein the pump delivers a predetermined amount of drug for a predetermined period of time, such as 30 minutes, 90 minutes, or a time period spanning the length of the treatment regimen.

The term “subcutaneous bolus” refers to drug administration beneath the skin of an animal or human patient, where bolus drug delivery is less than approximately 15 minutes; in another aspect, less than 5 minutes, and in still another aspect, less than 60 seconds. In yet even another aspect, administration is within a pocket between the skin and underlying tissue, where the pocket may be created by pinching or drawing the skin up and away from underlying tissue.

The term “therapeutically effective amount” is used to refer to an amount of an active agent having beneficial patient outcome, for example, a growth arrest effect or causes the deletion of the cell. In one aspect, the therapeutically effective amount has apoptotic activity, or is capable of inducing cell death. In another aspect, the therapeutically effective amount refers to a target serum concentration that has been shown to be effective in, for example, slowing disease progression. Efficacy can be measured in conventional ways, depending on the condition to be treated. For example, in neoplastic diseases or disorders characterized by cells expressing CD40, efficacy can be measured by assessing the time to disease progression (TTP), or determining the response rates (RR).

The terms “treatment” and “therapy” and the like, as used herein, are meant to include therapeutic as well as prophylactic, or suppressive measures for a disease or disorder leading to any clinically desirable or beneficial effect, including but not limited to alleviation or relief of one or more symptoms, regression, slowing or cessation of progression of the disease or disorder. Thus, for example, the term treatment includes the administration of an agent prior to or following the onset of a symptom of a disease or disorder thereby preventing or removing one or more signs of the disease or disorder. As another example, the term includes the administration of an agent after clinical manifestation of the disease to combat the symptoms of the disease. Further, administration of an agent after onset and after clinical symptoms have developed where administration affects clinical parameters of the disease or disorder, such as the degree of tissue injury or the amount or extent of metastasis, whether or not the treatment leads to amelioration of the disease, comprises “treatment” or “therapy” as used herein.

The term “package insert” is used to refer to instructions customarily included in commercial packages of therapeutic products, that contain information about the indications, usage, administration, contraindications and/or warnings concerning the use of such therapeutic products.

The abbreviation “AFP” refers to dimethylvaline-valine-dolaisoleuine-dolaproine-phenylalanine-p-phenylenediamine.

The abbreviation “MMAE” refers to monomethyl auristatin E.

The abbreviation “AEB” refers to an ester produced by reacting auristatin E with paraacetyl benzoic acid.

The abbreviation “AEVB” refers to an ester produced by reacting auristatin E with benzoylvaleric acid.

The abbreviation “MMAF” refers to dovaline-valine-dolaisoleunine-dolaproine-phenylalanine.

### CD40 Binding Agents

Described and disclosed herein are methods of using CD40 antibodies and other binding agents that specifically bind to CD40 (referred to as CD40 binding agents). The CD40 antibodies and binding agents can deliver a stimulatory signal to human B cells, enhance the interaction between CD40 and CD40L, and have in vivo anti-neoplastic activity. The CD40 antibodies and other CD40 binding agents can arrest the growth of cancer cells, cause the deletion of cells expressing CD40 or otherwise induce or cause a cytotoxic or cytostatic effect on target cells. The CD40 antibodies and other CD40 binding agents can be used in the treatment of a variety of diseases or disorders characterized by the proliferation of cells expressing the CD40 surface antigen.

In some aspects, the CD40 binding agent increases the binding of CD40 ligand to CD40 by at least 45%, by at least 50%, by at least 60% or by at least 75%. A method of determining increases in binding of CD40 ligand to CD40 are disclosed in U.S. Pat. No. 6,838,261 (the disclosure of which is incorporated by reference herein).

In various embodiments, the CD40 binding agent can, for example, block proliferation or otherwise arrest the growth of a cancer cell or cause its depletion, death, or otherwise its deletion, for example, through binding the CD40 surface antigen.

The CD40 binding agents (e.g., CD40 antibodies) each include at least a portion that specifically recognizes an epitope CD40, typically human CD40. In some embodiments the CD40 antibody or other CD40 binding agent includes an antigen-binding fragment of an antibody that specifically binds to CD40. In some embodiments, the CD40 binding agent competes for binding to CD40 with monoclonal antibody (mAb) S2C6. In some embodiments, CD40 binding agents include an antigen-binding fragment of a humanized CD40 antibody that binds to CD40 (e.g., human CD40 or a variant thereof). The CD40 binding agents can be optionally conjugated with or fused to a cytotoxic or chemotherapeutic agent. In aspects where the CD40 binding agent binds to the CD40 surface antigen and causes depletion of the CD40 expressing cell types, binding is generally characterized by homing to the CD40 surface antigen cell in vivo. Suitable binding agents bind the CD40 antigen with sufficient affinity and/or avidity such that the CD40 binding agent is useful as a therapeutic agent by specifically targeting a cell expressing the antigen.

The S2C6 antibody has been described, for example, by Paulie et al., 1984, Cancer Immunol. Immunother. 17:165-179.) The S2C6 antibody has been shown to exert an agonist activity on human peripheral B cells as demonstrated by the antibody's ability to stimulate primary B cell proliferation in a dose dependent manner (see, e.g., Paulie et al., 1989, J. Immunol. 142:590-595), as well as anti-neoplastic activity in vivo (see, e.g., U.S. Pat. No. 6,838,261).

In one aspect, the CD40 binding agents comprise all or a portion of monoclonal antibody S2C6 (the light chain and/or heavy chain, or light chain CDR 1 (SEQ ID NO:25) and/or 2 (SEQ ID NO:26), and/or heavy chain CDR 1 (SEQ ID NO:30), 2 (SEQ ID NO:31), and/or 3 (SEQ ID NO:32), or light chain CDR3 (SEQ ID NO:27) in combination with any of the other CDRs and/or one or more of the four heavy chain and four light chain framework regions, provided that such molecules are not native mAb S2C6 as deposited with the ATCC and assigned accession number PTA-10 or the heavy or light chain thereof. Such molecules may differ from S2C6 in sequence and/or in post-translational modification (glycosylation, amidation, peptide bonding or cross-linking to a non-S2C6 sequence, etc.).

In various specific embodiments, the CD40 binding agent immunospecifically binds CD40 (or when multimerized immunospecifically binds CD40), competes with native S2C6 for binding to CD40, and/or increases the binding of CD40 ligand to CD40 by at least 45%, 50%, 60% or 65%. Nucleic acids encoding such molecules, e.g., S2C6 fragments or derivatives, are also within the scope of the invention, as well as nucleic acids encoding native mAb S2C6. Production of the foregoing proteins, e.g., by recombinant methods, is provided.

Other embodiments provide CD40 binding agents such as proteins and derivatives including but not limited to fusion/chimeric proteins which are functionally active, i.e., which are capable of displaying one or more known functional activities associated with a full-length S2C6 mAb. Such functional activities include but are not limited to the ability to bind CD40, delivery of a stimulatory signal to the CD40 signaling pathway (e.g., so as to cause B cell proliferation), potentiation of the interaction of CD40L with CD40; ability to inhibit tumor growth; and ability to induce an immune response.

Antibodies to CD40 comprising S2C6, its derivatives and analogs include humanized antibodies, single chain antibodies, bispecific antibodies; and antibodies conjugated to therapeutic agents such as chemotherapeutic agents or biological response modifiers. Such antibodies include but are not limited to monoclonal, humanized, chimeric, single chain, bispecific, Fab fragments, F(ab′)₂ fragments, single chain Fv fragments, scFv-Fc fragments, minibodies, maxibodies, diabodies, tribodies, tetrabodies, fragments produced by a Fab expression library, anti-idiotypic (anti-Id) antibodies, and epitope-binding fragments of any of the above.

For preparation of additional monoclonal antibodies to CD40, any technique that provides for the production of antibody molecules by continuous cell, lines in culture may be used. These include but are not limited to the hybridoma technique of Kohler and Milstein (1975, Nature 256:495-497; and U.S. Pat. No. 4,376,110), the human B-cell hybridoma technique (Kozbor et al., 1983, Immunology Today 4:72; Cole et al., 1983, Proc. Natl. Acad. Sci. USA 80:2026-2030), and the EBV-hybridoma technique to produce human monoclonal antibodies (Cole et al., 1985, Monoclonal Antibodies and Cancer Therapy, Alan R. Liss, Inc., pp. 77-96). Such antibodies or other anti-CD40 antibodies available in the art may, e.g., be used as the basis from which to clone and thus supply a complementary light chain if a S2C6 heavy chain is to be recombinantly expressed (the two chains may be recombinantly expressed in the same cell or combined in vitro after separate expression and purification); alternatively, a light chain from an antibody of any specificity may be used. Nucleic acids (e.g., a plasmid) encoding an S2C6 heavy chain or encoding a molecule comprising an S2C6 heavy chain variable domain can be transfected into a cell expressing an antibody light chain or molecule comprising an antibody light chain, for expression of a multimeric protein; the antibody light chain can be recombinant or non-recombinant, and may or may not have anti-CD40 specificity. Alternatively, S2C6 heavy chains or molecules comprising the variable region thereof or a CDR thereof can optionally be expressed and used without the presence of a complementary light chain or light chain variable region. In various embodiments, the invention provides a S2C6 heavy chain with CD40 binding affinity, or a molecule consisting of or (alternatively) comprising one or more copies of heavy chain CDR 1, 2, and/or 3 (SEQ ID NO:30, 31 or 32, respectively) or a protein (peptide or polypeptide) the sequence of which consists of, or comprises, one or more copies of CDR 1, 2 or 3. In a specific embodiment, such a protein can be N or C-terminal modified, e.g., by C-terminal amidation or N-terminal acetylation.

In addition, techniques developed for the production of “chimeric antibodies” (Morrison et al., 1984, Proc. Natl. Acad. Sci. 81:6851-6855; Neuberger et al., 1984, Nature 312:604-608; Takeda et al., 1985, Nature 314:452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region-derived from a murine mAb and a human immunoglobulin constant region. (See, e.g., Cabilly et al., U.S. Pat. No. 4,816,567; and Boss et al., U.S. Pat. No. 5,816,397.) In a specific embodiment, the chimeric antibody comprises a variable domain of monoclonal antibody S2C6 secreted by the hybridoma as deposited with the ATCC and assigned accession number PTA-110, and a human constant region. In specific embodiments the variable domain of the chimeric antibody comprises the S2C6 V_{L} (SEQ ID NO:24) and/or the S2C6 V_{H} (SEQ ID NO:29).

In another aspect, the CD40 binding agent can be a humanized CD40 antibody. Techniques have been developed for the production of humanized antibodies. (See, e.g., Queen, U.S. Pat. No. 5,585,089; and Winter, U.S. Pat. No. 5,225,539.) An immunoglobulin light or heavy chain variable region consists of a “framework” region interrupted by three hypervariable regions, referred to as complementarity-determining regions (CDRs). The extent of the framework region and CDRs have been precisely defined (see, “Sequences of Proteins of Immunological Interest”, Kabat, E. et al., U.S. Department of Health and Human Services (1983)). Briefly, humanized antibodies are antibody molecules from non-human species having one or more CDRs from the non-human species and framework regions from a human immunoglobulin molecule.

In some embodiments, the CD40 binding agent is an antibody or derivative thereof comprising a heavy or light chain variable domain, said variable domain comprising (a) a set of three complementarity-determining regions (CDRs), in which said set of CDRs are from monoclonal antibody S2C6, and (b) a set of four framework regions, in which said set of framework regions differs from the set of framework regions in monoclonal antibody S2C6, and in which said antibody or derivative thereof inimunospecifically binds CD40. Preferably, the set of framework regions is from a human monoclonal antibody, e.g., a human monoclonal antibody that does not bind CD40.

In a specific embodiment, provided is an antibody or derivative thereof comprising a light chain variable domain, said variable domain comprising (a) a set of three complementarity-determining regions (CDRs), in which said set of CDRs comprises SEQ ID NO:25 or SEQ ID NO:26, and (b) a set of four framework regions, in which said set of framework regions differs from the set of framework regions in the light chain of monoclonal antibody S2C6, and in which said antibody or derivative thereof immunospecifically binds CD40.

In a specific embodiment, encompassed is an antibody or derivative thereof comprising a heavy chain variable domain, said variable domain comprising (a) a set of three complementarity-determining regions (CDRs), in which said set of CDRs comprises SEQ ID NO:30, SEQ ID NO:31, or SEQ ID NO:32, and (b) a set of four framework regions, in which said set of framework regions differs from the set of framework regions in the heavy chain of monoclonal antibody S2C6, and in which said antibody or derivative thereof immunospecifically binds CD40.

In specific embodiments, the human framework region amino acids are derived from human consensus sequences for the heavy chain subgroup III variable domain and the kappa light chain variable as described in U.S. Pat. No. 6,037,454. The humanized CD40 antibodies optionally include specific amino acid substitutions in the consensus framework regions.

The specific substitution of amino acid residues in these framework positions can improve various aspects of antibody performance including binding affinity and/or stability, over that demonstrated in humanized antibodies formed by “direct swap” of CDls or HVLs into the human consensus framework regions, as shown in the examples below.

In some embodiments, the humanized CD40 antibodies disclosed herein comprise at least a heavy or light chain variable domain comprising the CDRs or HVLs of the murine monoclonal antibody S2C6 and the FRs of the human consensus heavy and light chain variable domains having the specific substitutions described in Table 3 of Example 1. An alignment of the variable heavy chain amino acid sequences having substitutions and variable light chain amino acid sequences having substitutions are shown in Tables 3 and 4, respectively. These sequences include a heavy chain variable domain having the amino acid sequence of SEQ ID NO:3 and a light chain variable domain having the amino acid sequence of SEQ ID NO:14.

In certain embodiments, the humanized CD40 antibody is an antibody fragment. Various techniques have been developed for the production of antibody fragments. Fragments can be derived via proteolytic digestion of intact antibodies (see, e.g., Morimoto et al., 1992, Journal of Biochemical and Biophysical Methods 24:107-117; and Brennan et al., 1985, Science 229:81). Alternatively, the fragments can be produced directly in recombinant host cells. For example, Fab′-SH fragments can be directly recovered from E. coli and chemically coupled to form F(ab′)₂ fragments (see, e.g., Carter et al., 1992, Bio/Technology 10:163-167). By another approach, F(ab′)₂ fragments can be isolated directly from recombinant host cell culture. Other techniques for the production of antibody fragments will be apparent to the skilled practitioner.

Certain embodiments include an F(ab′)₂ fragment of a humanized CD40 antibody comprising a heavy chain variable domain amino acid sequence and a light chain variable domain amino acid sequence of SEQ ID NO:3 and SEQ ID NO:14, respectively; SEQ ID NO:4 and SEQ ID NO:14, respectively; SEQ ID NO:5 and SEQ ID NO:14, respectively; SEQ ID NO:6 and SEQ ID NO:14, respectively; SEQ ID NO:7 and SEQ ID NO:14, respectively; SEQ ID NO:8 and SEQ ID NO:14, respectively; SEQ ID NO:9 and SEQ ID NO:14, respectively; SEQ ID NO:6 and SEQ ID NO:15, respectively; SEQ ID NO:6 and SEQ ID NO:16, respectively; SEQ ID NO:7 and SEQ ID NO:16, respectively; SEQ ID NO:10 and SEQ ID NO:14, respectively; SEQ ID NO:11 and SEQ ID NO:14, respectively; SEQ ID NO:10 and SEQ ID NO:16, respectively; or SEQ ID NO:11 and SEQ ID NO:16, respectively. Such embodiments can include an intact antibody comprising such an F(ab′)₂.

Some embodiments include a CD40 binding agent having a heavy chain variable region amino acid sequence that is at least 80%, at least 90%, at least 95%, at least 98%, or at least 99% identical to the amino acid sequence of SEQ ID NO:3, SEQ ID NO:4, SEQ ID NO:5, SEQ ID NO:6, SEQ ID NO:7, SEQ ID NO:8, SEQ ID NO:9, SEQ ID NO:10, or SEQ ID NO:11. Some embodiments include a CD40 binding agent having a light chain variable domain amino acid sequence that is at least 80%, at least 90%, at least 95%, at least 98%, or at least 99% identical to the amino acid sequence of SEQ ID NO:14, SEQ ID NO:15, or SEQ ID NO:16.

Some embodiments, a CD40 binding agent having a heavy chain variable domain and a light chain variable region, each including an amino acid sequence that is at least 80%, at least 90%, at least 95%, at least 98%, or at least 99% identical to the amino acid sequence of SEQ ID NO:3 and SEQ ID NO:14, respectively; SEQ ID NO:4 and SEQ ID NO:14, respectively; SEQ ID NO:5 and SEQ ID NO:14, respectively; SEQ ID NO:6 and SEQ ID NO:14, respectively; SEQ ID NO:7 and SEQ ID NO:14, respectively; SEQ ID NO:8 and SEQ ID NO:14, respectively; SEQ ID NO:9 and SEQ ID NO:14, respectively; SEQ ID NO:6 and SEQ ID NO:15, respectively; SEQ ID NO:6 and SEQ ID NO:16, respectively; SEQ ID NO:7 and SEQ ID NO:16, respectively; SEQ ID NO:10 and SEQ ID NO:14, respectively; SEQ ID NO:11 and SEQ ID NO:14, respectively; SEQ ID NO:10 and SEQ ID NO:16, respectively; or SEQ ID NO:11 and SEQ ID NO:16, respectively.

Some further embodiments include a CD40 binding agent having a heavy chain variable domain and a light chain variable domain, each including an amino acid sequence that is at least 80%, at least 90%, at least 95%, at least 98%, or at least 99% identical to the amino acid sequence of SEQ ID NO:7 and SEQ ID NO:14, respectively; SEQ ID NO:6 and SEQ ID NO:16, respectively; SEQ ID NO:7 and SEQ ID NO:16, respectively; SEQ ID NO:10 and SEQ ID NO:14, respectively; SEQ ID NO:11 and SEQ ID NO:14, respectively; SEQ ID NO:10 and SEQ ID NO:16, respectively; and SEQ ID NO: 11 and SEQ ID NO:16, respectively.

Additional embodiments include a CD40 binding agent having a heavy chain variable region and a light chain variable region, each including an amino acid sequence that is at least 80%, at least 90%, at least 95%, at least 98%, or at least 99% identical to the amino acid sequence of SEQ ID NO:7 and SEQ ID NO:14, respectively; SEQ ID NO:6 and SEQ ID NO:16, respectively; SEQ ID NO:10 and SEQ ID NO:16, respectively; and SEQ ID NO:11 and SEQ ID NO:16, respectively.

Other embodiments include a CD40 binding agent having a heavy chain variable region and a light chain variable region, each including an amino acid sequence that is at least 80%, at least 90%, at least 95%, at least 98%, or at least 99% identical to the amino acid sequence of SEQ ID NO:10 and SEQ ID NO:16, respectively.

Other embodiments include a F(ab′)₂ fragment of a humanized CD40 antibody comprising a heavy chain variable domain amino acid sequence and a light chain variable domain amino acid sequence of SEQ ID NO:7 and SEQ ID NO:14, respectively; SEQ ID NO:6 and SEQ ID NO:16, respectively; SEQ ID NO:7 and SEQ ID NO:16, respectively; SEQ ID NO:10 and SEQ ID NO:14, respectively; SEQ ID NO:11 and SEQ ID NO:14, respectively; SEQ ID NO:10 and SEQ ID NO:16, respectively; and SEQ ID NO:11 and SEQ ID NO:16, respectively.

Yet other embodiments include a F(ab′)₂ fragment of a humanized CD40 antibody comprising a heavy chain variable domain amino acid sequence and a light chain variable domain amino acid sequence of SEQ ID NO:7 and SEQ ID NO:14, respectively; SEQ ID NO:6 and SEQ ID NO:16, respectively; SEQ ID NO:10 and SEQ ID NO:16, respectively; and SEQ ID NO:11 and SEQ ID NO:16, respectively.

Some embodiments include a F(ab′)₂ fragment of a humanized CD40 antibody that contains a heavy chain variable domain comprising the amino acid sequence of SEQ ID NO:10 and a light chain variable domain comprising the amino acid sequence of SEQ ID NO:16.

As used herein, the terms “identical” or “percent identity,” in the context of two or more polypeptide sequences, refer to two or more sequences or subsequences that are the same or have a specified percentage of amino acid residues that are the same, when compared and aligned for maximum correspondence. To determine the percent identity, the sequences are aligned for optimal comparison purposes (e.g., gaps can be introduced in the sequence of a first amino acid sequence for optimal alignment with a second amino or sequence). The amino acid residues at corresponding amino acid positions are then compared. When a position in the first sequence is occupied by the same amino acid residue as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In some embodiments, the two sequences that are compared are the same length after gaps are introduced within the sequences, as appropriate (e.g., excluding additional sequence extending beyond the sequences being compared). For example, when variable region sequences are compared, the leader and/or constant domain sequences are not considered. For sequence comparisons between two sequences, a “corresponding” CDR refers to a CDR in the same location in both sequences (e.g., CDR-H1 of each sequence).

As an alternative to humanization, human antibodies can be generated. For example, transgenic animals (e.g., mice) can be used that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., 1993, Proc. Natl. Acad. Sci. USA 90:2551; Jakobovits et al., 1993, Nature 362:255-258; Bruggermann et al., 1993, Year in Immuno. 7:33; and U.S. Pat. Nos. 5,591,669; 5,589,369; 5,545,807; 6,075,181; 6,150,584; 6,657,103; and 6,713,610.

Alternatively, phage display technology (see, e.g., McCafferty et al., 1990, Nature 348:552-553) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats; for their review see, e.g., Johnson and Chiswell, 1993, Current Opinion in Structural Biology 3:564-571. Several sources of V-gene segments can be used forphage display. Clackson et al., 1991, Nature 352:624-628 isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., 1991, J. Mol. BiaoL 222:581-597, or Griffith et al., 1993, EMBO J. 12:725-734. See also U.S. Pat. Nos. 5,565,332 and 5,573,905. As discussed above, human antibodies may also be generated by in vitro activated B cells (see U.S. Pat. Nos. 5,567,610 and 5,229,275).

In other embodiments, techniques described for the production of single chain antibodies (U.S. Pat. No. 4,946,778; Bird, 1988, Science 242:423-426; Huston et al., 1988, Proc. Natl. Acad. Sci. USA 85:5879-5883; and Ward et al., 1989, Nature 334:544-546) can be adapted to produce single chain antibodies using S2C6 sequences. Single chain antibodies are formed by linking the heavy and light chain fragments of the Fv region via an amino acid bridge, resulting in a single chain polypeptide. In a specific embodiment, the single chain antibody comprises the amino acid sequences as depicted in SEQ ID NOS:24 and 29, respectively).

In a specific embodiment, the antibody to a CD40 polypeptide, peptide or other derivative, or analog thereof comprising all or a portion of SEQ ID NO:23 or SEQ ID NO:28 is a bispecific antibody (see generally, e.g. Fanger and Drakeman, 1995, Drug News and Perspectives 8:133-137). Such a bispecific antibody is genetically engineered to recognize both (1) an epitope and (2) one of a variety of “trigger” molecules, e.g., Fc receptors on myeloid cells, and CD3 and CD2 on T cells, that have been identified as being able to cause a cytotoxic T-cell to destroy a particular target. Such bispecific antibodies can be prepared either by chemical conjugation, hybridoma, or recombinant molecular biology techniques known to the skilled artisan. In a specific embodiment, the bispecific antibody contains a molecule comprising the S2C6 heavy or light chain variable domain or a CDR sequence thereof, which molecule has the structure of an antibody heavy or light chain but which differs from the native S2C6 heavy or light chain (e.g., by having amino acid substitution(s) in the framework region or a human constant domain).

Other antibody fragments that retain the ability to recognize CD40 may be generated by known techniques. For example, such fragments include but are not limited to: F(ab′)₂ fragments, which can be produced by pepsin digestion of the antibody molecule, and F(ab′) fragments, which can be generated by reducing the disulfide bridges of the F(ab′)₂ fragments. Alternatively, Fab expression libraries may be constructed (Huse et al., 1989, Science 246:1275-1281) to allow rapid and easy identification of monoclonal Fab fragments with the desired specificity. Other antibodies that can be generated include single chain Fv fragments, scFv-Fc fragments, minibodies, maxibodies, diabodies, tribodies, tetrabodies, anti-idiotypic (anti-Id) antibodies, and epitope-binding fragments of any of the above.

In some embodiments, the antibody or antibody fragment includes a constant region that mediates effector function. The constant region can provide antibody-dependent cellular cytotoxicity (ADCC), antibody-dependent cellular phagocytosis (ADCP) and/or complement-dependent cytotoxicity (CDC) responses against a CD40-expressing target cell. The effector domain(s) can be, for example, an Fc region of an Ig molecule. Typically, the CD40 binding agent recruits and/or activates cytotoxic white blood cells (e.g., natural killer (NK) cells, phagocytotic cells (e.g., macrophages), and/or serum complement components).

The effector domain of an antibody can be from any suitable vertebrate animal species and isotypes. The isotypes from different animal species differ in their abilities to mediate effector functions. For example, the ability of human immunoglobulin to mediate CDC and ADCC/ADCP is generally in the order of IgM≈IgG₁≈IgG₃≈IgG₂>IgG₄ and IgG₁≈IgG₃≈IgG₂/IgM/IgG₄, respectively. Murine immunoglobulins mediate CDC and ADCC/ADCP generally in the order of murine IgM≈IgG₃>>IgG_{2b}>IgG₂ₐ>>IgG₁ and IgG_{2b}>IgG₂ₐ>IgG₁>>IgG₃, respectively. In another example, murine IgG₂ₐ mediates ADCC while both murine IgG₂ₐ and IgM mediate CDC.

### CD40 Binding Agent Modifications

In other aspects, derivatives (including but not limited to fragments), analogs, and molecules of CD40 binding agents (e.g., monoclonal antibodies or fragments thereof derived from mAb) are provided. Nucleic acids encoding S2C6 protein derivatives and protein analogs are also provided. In particular aspects, the proteins, derivatives, or analogs are encoded by the sequence of SEQ ID NO:23 or SEQ ID NO:28.

The production and use of derivatives and analogs related to an S2C6 mAb are provided. In a specific embodiment, the derivative or analog is functionally active, i.e., capable of exhibiting one or more functional activities associated with a full-length, S2C6 mAb. As one example, such derivatives or analogs which have the desired binding specificity can be used in immunoassays, or therapeutically for inhibition of tumor growth, etc. A specific embodiment relates to an S2C6 mAb fragment that binds CD40 and potentiates binding of CD40L to CD40. Derivatives or analogs of an S2C6 protein can be tested for the desired activity by various immunoassays known in the art, including but not limited to competitive and non-competitive assay systems using techniques such as radioimmunoassays, enzyme linked immunosorbent assay (ELISA), “sandwich” immunoassays, Western blots, immunofluorescence assays, protein A assays, immunoelectrophoretic assays, etc.

In addition, assays known in the art can be used to detect or measure the ability to inhibit cell proliferation (e.g., inhibition of tumor cell growth) or ability to stimulate cell proliferation (e.g., proliferation of B cells) in vivo or in vitro.

In particular, S2C6 mAb derivatives can be made by altering S2C6 mAb sequences by substitutions, additions (e.g., insertions) or deletions that provide for functionally equivalent molecules. Due to the degeneracy of nucleotide coding sequences, other DNA sequences which encode substantially the same amino acid sequence as a nucleic acid encoding S2C6 mAb may be used in the practice of the present invention. These include but are not limited to nucleotide sequences comprising all or portions of an S2C6 mAb gene which is altered by the substitution of different codons that encode a functionally equivalent amino acid residue within the sequence, thus producing a silent change. Likewise, the S2C6 mAb derivatives include, but are not limited to, those containing, as a primary amino acid sequence, all or part of the amino acid sequence of an S2C6 mAb, including altered sequences in which functionally equivalent amino acid residues are substituted for residues within the sequence resulting in a silent change. For example, one or more amino acid residues within the sequence can be substituted by another amino acid of a similar polarity which acts as a functional equivalent, resulting in a silent alteration. Substitutions for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Such substitutions are generally understood to be conservative substitutions.

In a specific embodiment, a CD40 binding agent consisting of or comprising a fragment of an S2C6 mAb consisting of at least 10 (continuous) amino acids of the S2C6 mAb is provided. In other embodiments, the fragment consists of at least 20 or at least 50 amino acids of the S2C6 mAb. In specific embodiments, such fragments are not larger than 50, 75, 100, or 200 amino acids. Derivatives or analogs of S2C6 mAbs include but are not limited to those molecules comprising regions that are substantially homologous to an S2C6 mAb or fragment thereof (e.g., in various embodiments, at least 60% or 70% or 80% or 90% or 95% identity over an amino acid sequence of identical size with no insertions or deletions or when compared to an aligned sequence in which the alignment is done by a computer homology program known in the art) or whose encoding nucleic acid is capable of hybridizing to a coding S2C6 gene sequence, under high stringency, moderate stringency, or low stringency conditions.

Specifically, by way of example computer programs for determining homology may include but are not limited to TBLASTN, BLASTP, FASTA, TEASTA, and CLUSTALW (Pearson and Lipman, 1988, Proc. Natl. Acad. Sci. USA 85(8):2444-8; Altschul et al., 1990, J. Mol. Biol. 215(3):403-10; Thompson et al., 1994, Nucleic Acids Res. 22(22):4673-80; Higgins et al., 1996, Methods Enzymol. 266:383-402; Altschul et al., 1990, J. Mol. Biol. 215(3):403-10). Default parameters for each of these computer programs are well known and can be utilized.

Specifically Basic Alignment Search Tool (BLAST) (Altschul et al., 1990, J. Mol. Biol. 215:403-410, “The BLAST Algorithm; Altschul et al., 1997, Nuc. Acids Res. 25:3389-3402) is a heuristic search algorithm tailored to searching for sequence similarity which ascribes significance using the statistical methods of Karlin and Altschul 1990, Proc. Natl. Acad. Sci. USA 87:2264-68; 1993, Proc. Natl. Acad. Sci. USA 90:5873-77. Five specific BLAST programs perform the following tasks: 1) the BLASTP program compares an amino acid query sequence against a protein sequence database; 2) the BLASTN program compares a nucleotide query sequence against a nucleotide sequence database; 3) the BLASTX program compares the six-frame conceptual translation products of a nucleotide query sequence (both strands) against a protein sequence database; 4) the TBLASTN program compares a protein query sequence against a nucleotide sequence database translated in all six reading frames (both strands); 5) the TBLASTX program compares the six-frame translations of a nucleotide query sequence against the six-frame translations of a nucleotide sequence database.

Smith-Waterman (database: European Bioinformatics Institute (Smith-Waterman, 1981, J. Mol. Biol. 147:195-197) is a mathematically rigorous algorithm for sequence alignments.

FASTA (see Pearson et al, 1988, Proc. Natl. Acad. Sci. USA 85:2444-2448) is a heuristic approximation to the Smith-Waterman algorithm. For a general discussion of the procedure and benefits of the BLAST, Smith-Waterman and FASTA algorithms see Nicolas et al., 1998, “A Tutorial on Searching Sequence Databases and Sequence Scoring Methods” and references cited therein.

The S2C6 mAb derivatives and analogs can be produced by various methods known in the art. The manipulations which result in their production can occur at the gene or protein level. For example, a cloned S2C6 gene sequence can be modified by any of numerous strategies known in the art (Sambrook et al., 1989, Molecular Cloning, A Laboratory Manual, 2d ed., Cold Spring Harbor Laboratory Press, Cold Spring Harbor, N.Y.). The sequence can be cleaved at appropriate sites with restriction endonuclease(s), followed by further enzymatic modification if desired, isolated, and ligated in vitro. In the production of a modified gene encoding a derivative or analog of the S2C6 protein, care should be taken to ensure that the modified gene remains within the same translational reading frame as the native protein, uninterrupted by translational stop signals, in the gene region where the desired S2C6 protein activity is encoded.

Additionally, an S2C6 nucleic acid sequence can be mutated in vitro or in vivo, to create and/or destroy translation, initiation, and/or termination sequences, or to create variations in coding regions and/or to form new restriction endonuclease sites or destroy preexisting ones, to facilitate further in vitro modification. Any technique for mutagenesis known in the art can be used, including but not limited to, chemical mutagenesis, in vitro site-directed mutagenesis (Hutchinson et al., 1978, J. Biol. Chem. 253:6551), PCR with primers containing a mutation, etc.

Manipulations of an S2C6 mAb sequence may also be made at the protein level. Included are S2C6 protein fragments or other derivatives or analogs which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited to specific chemical cleavage by cyanogen bromide, trypsin, chymotrypsin, papain, V8 protease, NaBH4, acetylation, formylation, oxidation, reduction, metabolic synthesis in the presence of tunicamycin, etc.

In addition, analogs and derivatives of an S2C6 mAb can be chemically synthesized. For example, a peptide corresponding to a portion of an S2C6 mAb which comprises the desired domain, or which mediates the desired activity in vitro, can be synthesized by use of a peptide synthesizer. Furthermore, if desired, nonclassical amino acids or chemical amino acid analogs can be introduced as a substitution or addition into the S2C6 mAb sequence. Non-classical amino acids include but are not limited to the D-isomers of the common amino acids, alpha-amino isobutyric acid, 4-aminobutyric acid, Abu, 2-amino butyric acid, gamma-Abu, epsilon-Ahx, 6-amino hexanoic acid, Aib, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, beta-alanine, fluoro-amino acids, designer amino acids such as beta-methyl amino acids, Calpha-methyl amino acids, Nalpha-methyl amino acids, and amino acid analogs in general. Furthermore, the amino acid can be D (dextrorotary) or L (levorotary).

The CD40 binding agents also can include modifications of a CD40 antibody or antigen-binding fragment thereof. For example, it may be desirable to modify the antibody with respect to effector function, so as to enhance the effectiveness of the antibody in treating cancer. One such modification is the introduction of cysteine residue(s) into the Fc region, thereby allowing interchain disulfide bond formation in this region. The homodimeric antibody thus generated can have improved internalization capability and/or increased complement-mediated cell killing and/or antibody-dependent cellular cytotoxicity (ADCC). See, for example, Caron et al., 1992, J. Exp Med. 176:1191-1195; and Shopes, 1992, J. Immunol. 148:2918-2922. Homodimeric antibodies having enhanced anti-tumor activity can also be prepared using heterobifunctional cross-linkers as described in Wolff et al., 1993, Cancer Research 53: 2560-2565. Alternatively, an antibody can be engineered to contain dual Fc regions, enhancing complement lysis and ADCC capabilities of the antibody. See Stevenson et al., 1989, Anti-Cancer Drug Design 3: 219-230.

Antibodies with improved ability to support ADCC have been generated by modifying the glycosylation pattern of their Fe region. This is possible since antibody glycosylation at the asparagine residue, N297, in the C_{H}2 domain is involved in the interaction between IgG and Fcγ receptors prerequisite to ADCC. Host cell lines have been engineered to express antibodies with altered glycosylation, such as increased bisecting N-acetylglucosamine or reduced fucose. Fucose reduction provides greater enhancement to ADCC activity than does increasing the presence of bisecting N-acetylglucosamine. Moreover, enhancement of ADCC by low fucose antibodies is independent of the FcγRIIIa V/F polynorphism.

Modifying the amino acid sequence of the Fc region of antibodies is an alternative to glycosylation engineering to enhance ADCC. The binding site on human IgG₁ for Fcγ receptors has been determined by extensive mutational analysis. This led to the generation of humanized IgG₁ antibodies with Fc mutations that increase the binding affinity for FcγRIIIa and enhance ADCC in vitro. Additionally, Fc variants have been obtained with many different permutations of binding properties, e.g., improved binding to specific FcγR receptors with unchanged or diminished binding to other FcγR receptors.

In some embodiments, the Fc region can be modified as described in U.S. Patent Application Publication Nos. 2006-0003412 and 2006-0008883, the disclosures of which are incorporated by reference herein.

In other specific embodiments, the CD40 binding agent (e.g., a S2C6 mAb, fragment, analog, or derivative) may be expressed as a fusion, or chimeric protein product (comprising the protein, fragment, analog, or derivative joined via a peptide bond to a heterologous protein sequence of a different protein, also referred to herein as an immunoconjugate). The heterologous protein sequence can comprise a biological response modifier, including but not limited to interferon-alpha, interferon gamma, interleukin-2, interleukin-4, interleukin-6, and tumor necrosis factor, or a functionally active portion thereof. Alternatively, the heterologous protein sequence can comprise enzymes such as beta-lactamase or carboxylesterases or toxins such as bryodin 1, Pseudomonas exotoxin A, or gelonin, or a functionally active portion thereof. Additionally, the CD40 binding agent protein can be chemically linked to a chemotherapeutic agents, including but not limited to alkylating agents (e.g., nitrogen mustards, nitrosoureas, triazenes); antimetabolites (e.g., folic acid analogs, pyrimidine analogs, purine analogs); natural products (e.g., antibiotics, enzymes, biological response modifiers); miscellaneous agents (e.g. substituted urea, platinum coordination complexes); and hormones and antagonists (e.g., estrogens, androgens, antiandrogen, gonadotropin releasing hormone analog); or functionally active portion thereof (see, e.g., Goodman and Gilman, The Pharmacological Basis of Therapeutics, Ninth Edition, McGraw-Hill, pp. 1225-1287, 1996). Other nonlimiting examples of heterologous proteins and chemotherapeutic agents suitable for the production of a S2C6 chimeric protein product are known the skilled artisan. Such a chimeric product can be made by ligating the appropriate nucleic acid sequences encoding the desired amino acid sequences to each other by methods known in the art, in the proper coding frame, and expressing the chimeric product by methods commonly known in the art. Alternatively, such a chimeric product may be made by protein synthetic techniques, e.g., by use of a peptide synthesizer. In different embodiments, the heterologous protein sequence can be covalently bound to the S2C6-related sequences by other than a peptide bond, e.g., by use of chemical crosslinking agents well known in the art.

In a specific embodiment, a CD40 binding agent is a chimeric or fusion protein comprising an S2C6 mAb or fragment thereof (preferably consisting of at least a domain or motif of the S2C6 mnAb, or at least 10, 50 or 100 amino acids of the S2C6 mAb) joined at its amino- or carboxy-terminus via a peptide bond to an amino acid sequence of a different protein. In a specific embodiment, the different protein is a toxin, enzyme or biological response modifier.

In specific embodiments, the amino acid sequence of the different protein is at least 6, 10, 20 or 30 continuous amino acids of the different protein or a portion of the different protein that is functionally active. In one embodiment, such a chimeric protein is produced by recombinant expression of a nucleic acid encoding the protein (comprising an S2C6 mAb-coding sequence joined in-frame to a coding sequence for a different protein). Such a chimeric product can be made by ligating the appropriate nucleic acid sequences encoding the desired amino acid sequences to each other by methods known in the art, in the proper coding frame, and expressing the chimeric product by methods commonly known in the art. Alternatively, such a chimeric product may be made by protein synthetic techniques, e.g., by use of a peptide synthesizer. Chimeric genes comprising portions of an S2C6 mAb gene fused to any heterologous protein-encoding sequences may be constructed. A specific embodiment relates to a chimeric protein comprising a fragment of an S2C6 mAb of at least 6 or 15 or 50 amino acids, or a fragment that displays one or more functional activities of the S2C6 mAb (e.g., comprising copies of one or more CDRs).

In a specific embodiment, the S2C6 mAb or derivative thereof is chemically linked to a chemotherapeutic drug including but not limited to doxorubicin, paclitaxel, docetaxel or an auristatin. Such a S2C6 mAb-drug conjugate can deliver the drug to cells expressing CD40. One or more drug molecules can be linked to the S2C6 mAb or derivative. Linkages include but are not limited to hydrazone, peptide or carbohydrate linkages.

Another aspect includes immunoconjugates comprising a CD40 binding agent (e.g., a CD40 antibody or fragment thereof) conjugated to a cytotoxic agent such as a chemotherapeutic agent, a toxin (e.g., an enzymatically active toxin of bacterial, fimgal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used to form useful immunoconjugates include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, the tricothecenes, and the like. A variety of radionuclides are available for the production of radioconjugated CD40 binding agents. Examples include ²¹²Bi, ¹³¹I, ¹³¹In, ⁹⁰Y, and ¹⁸⁶Re.

Conjugates of a CD40 binding agent and cytotoxic or chemotherapeutic agent can be made by known methods, using a variety of bifunctional protein coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., 1987, Science 238:1098. Carbon¹⁴-labeled 1-isothiocyanatobenzyl-3-methyldiethylene triaminepentaacetic acid (MX-DTPA) is an exemplary chelating agent for conjugation of radionucleotide to the antibody. See, e.g., International Publication WO 94/11026. Conjugates also can be formed with a cleavable linker, such as that disclosed in published EP Patent Application 0 624 377; the disclosure of which is incorporated by reference herein.

In another embodiment, the CD40 binding agent may be conjugated to a “receptor” (such as streptavidin) for utilization in tumor pretargeting. In this procedure, the antibody-receptor conjugate is administered to a patient, followed by removal of unbound conjugate from the circulation using a clearing agent and then administration of a “ligand” that selectively binds the receptor (e.g., avidin), the ligand being conjugated to a cytotoxic agent (e.g., a radionuclide).

The CD40 binding agents disclosed herein can also be formulated as immunoliposomes. Liposomes containing the antibody are prepared by methods known in the art, such as described in Epstein et al., 1985, Proc. Natl. Acad. Sci. USA 82:3688; Hwang et al., 1980, Proc. Natl. Acad. Sci. USA 77:4030; and U.S. Pat. Nos. 4,485,045 and 4,544,545. Liposomes having enhanced circulation time are disclosed, for example, in U.S. Pat. No. 5,013,556.

Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of an antibody disclosed herein can be conjugated to the liposomes as described in Martin et al., 1982, J. Biol. Chem. 257:286-288 via a disulfide interchange reaction. A chemotherapeutic agent (such as doxorubicin) is optionally contained within the liposome. See, e.g., Gabizon et al., 1989, J. National Cancer Inst. 81(19):1484.

In certain embodiments, it may be desirable to use an antibody fragment, rather than an intact antibody, to increase tumor penetration, for example. It may be desirable to modify the antibody fragment in order to increase its serum half life. This can be achieved, for example, by incorporation of a salvage receptor binding epitope into the antibody fragment. In one method, the appropriate region of the antibody fragment can be altered (e.g., mutated), or the epitope can be incorporated into a peptide tag that is then fused to the antibody fragment at either end or in the middle, for example, by DNA or peptide synthesis. See, e.g., WO 96/32478.

In other embodiments, covalent modifications of the CD40 binding agent are also included. They may be made by chemical synthesis or by enzymatic or chemical cleavage of the antibody, if applicable. Other types of covalent modifications of the antibody can be introduced into the molecule by reacting targeted amino acid residues of the antibody with an organic derivatizing agent that is capable of reacting with selected side chains or the amino- or carboxy-terminal residues.

Covalent modifications include modification of cysteinyl residues, histidyl residues, lysinyl and amino-terminal residues, arginyl residues, tyrosyl residues, carboxyl side groups (aspartyl or glutamyl), glutaminyl and asparaginyl residues, or seryl, or threonyl residues. Another type of covalent modification involves chemically or enzymatically coupling glycosides to the antibody.

Removal of any carbohydrate moieties present on the antibody can be accomplished chemically or enzymatically. Chemical deglycosylation is described by Hakimuddin et al., 1987, Arch. Biochem. Biophys. 259:52 and by Edge et al., 1981, Anal. Biochem., 118:131. Enzymatic cleavage of carbohydrate moieties on antibodies can be achieved by the use of a variety of endo- and exo-glycosidases as described by Thotakura et al., 1987, Meth. Enzymol. 138:350.

Another type of useful covalent modification comprises linking the antibody to one of a variety of nonproteinaceous polymers, e.g., polyethylene glycol, polypropylene glycol, or polyoxyalkylenes, in the manner set forth in one or more of U.S. Pat. No. 4,640,835, U.S. Pat. No. 4,496,689, U.S. Pat. No. 4,301,144, U.S. Pat. No. 4,670,417, U.S. Pat. No. 4,791,192 and U.S. Pat. No. 4,179,337.

### Therapeutic Uses

The CD40 binding agents are useful in the treatment of various disorders associated with the expression of CD40 as described herein.

The CD40 binding agents can be administered by any suitable means, including parenteral, subcutaneous, intraperitoneal, intrapulmonary, and intranasal, and, if desired for local immunomodulatory treatment, intralesional administration (including perfusing or otherwise contacting the graft with the antibody before transplantation). The CD40 binding agents can be administered, for example, as an infusion or as a bolus. Parenteral infusions include intramuscular, intravenous, intraarterial, intraperitoneal, or subcutaneous administration. In addition, the CD40 binding agents are suitably administered by pulse infusion, particularly with declining doses of the antibody. In one aspect, the dosing is given by injections, most preferably intravenous or subcutaneous injections, depending in part on whether the administration is brief or chronic.

For the prevention or treatment of disease, the appropriate dosage of antibody will depend on a variety of factors such as the type of disease to be treated, as defined above, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments.

The amount of the CD40 binding agent (e.g., a CD40 antibody) that is effective in the treatment or prevention of an immunological disorder or CD40-expressing cancer can be determined by standard clinical techniques. In addition, in vitro assays may optionally be employed to help identify optimal dosage ranges. The precise dose to be employed in the formulation will also depend on the route of administration, and the stage of immunological disorder or CD40-expressing cancer, and should be decided according to the judgment of the practitioner and each patient's circumstances. Effective doses may be extrapolated from dose-response curves derived from in vitro or animal model test systems.

In some embodiments, the dosage of the CD40 binding agent administered to a patient with a CD40-associated disorder, such as an immunological disorder or CD40-expressing cancer. The dosage is typically about 0.1 mg/kg to about 100 mg/kg of the patient's body weight. The dosage administered to a patient can be about 0.1 mg/kg to about 50 mg/kg, about 1 mg/kg to about 30 mg/kg, about 1 mg/kg to about 20 mg/kg, about 1 mg/kg to about 15 mg/kg, or about 1 mg/kg to about 10 mg/kg of the patient's body weight.

Exemplary doses include, but are not limited to, from 1 mg/kg to 100 mg/kg. In some embodiments, a dose is about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg or about 16 mg/kg. The dose can be administered, for example, daily, once per week (weekly), twice per week, thrice per week, four times per week, five times per week, six times per week, biweekly or monthly.

In specific embodiments, the dose is about 0.5 mg/kg/week, about 1 mg/kg/week, about 2 mg/kg/week, about 3 mg/kg/week, about 4 mg/kg/week, about 5 mg/kg/week, about 6 mg/kg/week, about 7 mg/kg/week, about 8 mg/kg/week, about 9 mg/kg/week, about 10 mg/kg/week, about 11 mg/kg/week, about 12 mg/kg/week, about 13 mg/kg/week, about 14 mg/kg/week, about 15 mg/kglweek or about 16 mg/kg/week. In some embodiments, the dose ranges from about 1 mg/kglweek to about 15 mg/kg/week. In some embodiments, the dose ranges from about 1 mg/kg/week to about 10 mg/kg/week. In some embodiments, the dose ranges from about 1 mg/kg/week to about 8 mg/kgtweek.

In other embodiments, the dose is at least about 4 mg/kg/week, at least about 5 mg/kg/week, at least about 6 mg/kg/week, at least about 7 mg/kg/week, or at least about 8 mg/kg/week. In yet other embodiments, the dose ranges from about 4 mg/kg/week to about 15 mg/kg/week. In some embodiments, the dose ranges from about 4 mg/kg/week to about 10 mg/kg/week. In some embodiments, the dose ranges from about 4 mg/kg/week to about 8 mg/kg/week.

In some embodiments, an escalating dosing schedule is used, in which the CD40 binding agent is administered in one or more cycles. Each cycle is typically followed by a non-dosing period of at least one week. Each dosing cycle can last at least one, at least two, at least three, at least four, or at least five weeks. Each non-dosing period can last at least one week, at least two weeks, at least three weeks or at least four weeks.

Each cycle includes an initial (first) dose, followed by a subsequent, higher dose. In some embodiments, the first dose is a sub-therapeutic or suboptimal dose, followed by a higher dose administered later. In other embodiments, the initial dose is a therapeutic dose, followed by a higher dose administered later. As used herein, a suboptimal dose refers to a dose that is less than the desired therapeutic dose. Optionally, the dosing levels can continue to increase (e.g., a third higher dose, a fourth higher dose, a fifth higher dose, etc.) until a desired (target) therapeutically effective dose is achieved.

The initial dose can be administered one, two, three, four, five, six or more times. The dosing interval can daily, every other day, every third day, every fourth day, every fifth, every sixth day or weekly. In some embodiments, the initial dose can be administered, for example, every two, every three, every four days or every five days. In exemplary embodiments, an initial dose can be about 0.5 mg/kg, about 1 mg/kg, about 2 mg/kg, about 3 mg/kg, about 4 mg/kg, about 5 mg/kg or about 6 mg/kg.

The subsequent doses are typically increased, as compared with the initial dose(s). For example, an initial dose can be about 0.5 mg/kg, about 1 mg/kg, about 2 mg/kg, about 3 mg/kg, about 4 mg/kg, about 5 mg/kg or about 6 mg/kg. The initial dose can optionally be repeated (i.e., administered a second time). The subsequent dose is greater than the initial dose and can be about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg or about 16 mg/kg. The subsequent doses can be administered every day, every two days, every three days, every four days, every five days, every six days, weekly, every seven days, every eight days, every nine days, every ten days, biweekly or monthly.

In some embodiments, an initial dose is administered on day one, followed by the second dose on day two, day three, day four or day five. A third dose optionally can be administered on day three, day four, day five, day six, day seven, day eight, day nine or day ten. Additional doses can be administered, for example, every three, four, five, six, seven, eight, nine or ten days.

In some embodiments, the dosing schedule is as follows: an initial dose of 1 mg/kg; optionally followed by a second dose of 1 mg/kg; a subsequent dose of 2 mg/kg; a subsequent dose of 3 mg/kg; and optionally at least one subsequent dose of 4 mg/kg, 5 mg/kg, 6 mg/kg, 7 mg/kg or 8 mg/kg. In other embodiments, the initial dose is 1 mg/kg, 2 mg/kg, 3 mg/kg, 4 mg/kg, 5 mg/kg or 6 mg/kg and the subsequent dose is the target therapeutic dose.

Exemplary dosing cycles include the following:

| Week 1 | | Week 2 | Week 3 | Week 4 | Week 5 |
|---|---|---|---|---|---|
| Day 1 | Day 4 | Day 8 | Day 15 | Day 22 | Day 29 |
| 1 mg/kg | 1 mg/kg | 2 mg/kg | 3 mg/kg | 3 mg/kg | 3 mg/kg |
| 1 mg/kg | 1 mg/kg | 2 mg/kg | 4 mg/kg | 4 mg/kg | 4 mg/kg |
| 1 mg/kg | 1 mg/kg | 2 mg/kg | 4 mg/kg | 6 mg/kg | 6 mg/kg |
| 1 mg/kg | 1 mg/kg | 2 mg/kg | 4 mg/kg | 6 mg/kg | 8 mg/kg |
| 1 mg/kg | 1 mg/kg | 4 mg/kg | 8 mg/kg | 8 mg/kg | 8 mg/kg |
| 1 mg/kg | 2 mg/kg | 4 mg/kg | 8 mg/kg | 8 mg/kg | 8 mg/kg |
| 2 mg/kg | 2 mg/kg | 4 mg/kg | 8 mg/kg | 8 mg/kg | 8 mg/kg |
| 2 mg/kg | 4 mg/kg | 8 mg/kg | 8 mg/kg | 8 mg/kg | 8 mg/kg |
| 4 mg/kg | 4 mg/kg | 8 mg/kg | 12 mg/kg | 12 mg/kg | 12 mg/kg |
| 4 mg/kg | 8 mg/kg | 12 mg/kg | 12 mg/kg | 12 mg/kg | 12 mg/kg |
| 8 mg/kg | 8 mg/kg | 16 mg/kg | 16 mg/kg | 16 mg/kg | 16 mg/kg |

In some embodiments, a patient receiving a cycle of a CD40 binding agent maintains a blood plasma level of at least 5 μg/ml after the initial dose and during the cycle. In some embodiments, a patient receiving a cycle of a CD40 binding agent maintains a blood plasma level of at least 10 μg/ml after the initial dose and during the cycle.

In some embodiments, the escalated dosing schedule reduces or prevents at least one adverse event associated with administration of the CD40 binding agent, as compared with the administration of a full therapeutic dose (e.g., 3, 4, 6, 8, 12, or 16 mg/kg). Such adverse effects can include, for example, hypotension, rash, headache, sinus headache, headache/aseptic meningitis, anorexia, increased body temperature, increased creatinine, urinary tract infection, upper respiratory infection, gait disturbance, fatigue, chills, vomiting, nausea, diarrhea, conjunctivitis, neutropenia, anemia or elevated liver transaminases. In some embodiments, the occurrence or frequency of Grade 3 adverse events is reduced. In some embodiments, the occurrence or frequency of Grade 2 adverse events is reduced. In some embodiments, the occurrence or frequency of Grade 1 adverse events is reduced. In some embodiments, the escalated dosing schedule reduces first-dose cytokine release in a patient, as compared to a patient not receiving an initial dose on the escalated dosing schedule (e.g., receiving instead a target therapeutic dose).

In some embodiments, the CD40 binding agent is co-administered with a therapeutic agent that reduces cytokine release. The therapeutic agent can be administered before, during, or after administration of the CD40 binding agent. For example, the CD40 binding agent can be co-administered with a therapeutic agent that reduces cytokine release (e.g., in the treatment of multiple myeloma or NHL). The therapeutic agent can be, for example, a steroid, such as a cortiocosterid (e.g., methylprednisolone), diphenyldramine, acetaminophen, rituximab, or other agent that inhibits or reduces cytokine release triggered by a dosage of a CD40 binding agent. For example, a therapeutic agent that reduces cytokine release can be administered before, concurrent with or after a dose (e.g., a first dose) of a CD40 binding agent.

In some embodiments, a CD40 binding agent is co-administered with rituximab (e.g., about 2 mg/kg) to a patient having Waldenstrom's macroglobulinemia. In some embodiments, a steroid (e.g., dexamethasone, prednisone, prednisolone or methylprednisolone) is administered prior to administration of a CD40 binding agent.

The CD40 binding agent will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the CD40 binding agent to be administered will be governed by such considerations, and is the minimum amount necessary to prevent, ameliorate, or treat the disorder associated with CD40 expression.

The CD40 binding agent need not be, but is optionally, formulated with one or more agents currently used to prevent or treat the disorder in question. The effective amount of such other agents depends on the amount of CD40 binding agent present in the formulation, the type of disorder or treatment, and other factors discussed above. These are generally used in the same dosages and with administration routes as used hereinbefore or about from 1 to 99% of the heretofore employed dosages.

### CD40-Associated Disorders

The CD40 binding agents are useful for treating or preventing a CD40-expressing cancer or an immunological disorder characterized by expression of CD40, e.g., by inappropriate activation of immune cells (e.g., lymphocytes or dendritic cells). Such expression of CD40 can be due to, for example, increased CD40 protein levels on the cells surface and/or altered antigenicity of the expressed CD40. Treatment or prevention of the immunological disorder, according to the methods described herein, is achieved by administering to a patient in need of such treatment or prevention an effective amount of the CD40 binding agent agent, whereby the agent (i) binds to activated immune cells that express CD40 and that are associated with the disease state and (ii) exerts a cytotoxic, cytostatic, or immunomodulatory effect on the activated immune cells.

Immunological diseases that are characterized by inappropriate activation of immune cells and that can be treated or prevented by the methods described herein can be classified, for example, by the type(s) of hypersensitivity reaction(s) that underlie the disorder. These reactions are typically classified into four types: anaphylactic reactions, cytotoxic (cytolytic) reactions, immune complex reactions, or cell-mediated immunity (CMI) reactions (also referred to as delayed-type hypersensitivity (DTH) reactions). (See, e.g., Fundamental Immunology (William E. Paul ed., Raven Press, N.Y., 3rd ed. 1993).)

Specific examples of such immunological diseases include the following: rheumatoid arthritis, autoimmune demyelinative diseases (e.g., multiple sclerosis, allergic encephalomyelitis), endocrine opthalmopathy, uveoretinitis, systemic lupus erythematosus, myasthenia gravis, Grave's disease, glomerulonephritis, autoimmune hepatological disorder, inflammatory bowel disease (e.g., Crohn's disease or ulcerative colitis), anaphylaxis, allergic reaction, Sjogren's syndrome, type I diabetes mellitus, primary biliary cirrhosis, Wegener's granulomatosis, fibromyalgia, polymyositis, dermatomyositis, inflammatory myositis, multiple endocrine failure, Schmidt's syndrome, autoimmune uveitis, Addison's disease, adrenalitis, thyroiditis, Hashimoto's thyroiditis, autoimmune thyroid disease, pernicious anemia, gastric atrophy, chronic hepatitis, lupoid hepatitis, atherosclerosis, subacute cutaneous lupus erythematosus, hypoparathyroidism, Dressler's syndrome, autoimmune thrombocytopenia, idiopathic thrombocytopenic purpura, hemolytic anemia, pemphigus vulgaris, pemphigus, dermatitis herpetiformis, alopecia arcata, pemphigoid, sclerodenna, progressive systemic sclerosis, CREST syndrome (calcinosis, Raynaud's phenomenon, esophageal dysmotility, sclerodactyl), and telangiectasia), male and female autoimmune infertility, ankylosing spondolytis, ulcerative colitis, mixed connective tissue disease, polyarteritis nedosa, systemic necrotizing vasculitis, atopic dernatitis, atopic rhinitis, Goodpasture's syndrome, Chagas' disease, sarcoidosis, rheumatic fever, asthma, recurrent abortion, anti-phospholipid syndrome, farmer's lung, erythema multiforme, post cardiotomy syndrome, Cushing's syndrome, autoimmune chronic active hepatitis, bird-fancier's lung, toxic epidermal necrolysis, Alport's syndrome, alveolitis, allergic alveolitis, fibrosing alveolitis, interstitial lung disease, erythema nodo sum, pyodenna gangreno sum, transfusion reaction, Takayasu's arteritis, polymyalgia rheumatica, temporal arteritis, schistosomiasis, giant cell arteritis, ascariasis, aspergillosis, Sampter's syndrome, eczema, lymphomatoid granulomatosis, Behcet's disease, Caplan's syndrome, Kawasaki's disease, dengue, encephalomyelitis, endocarditis, endomyocardial fibrosis, endophthalmitis, erythema elevatum et diutinum, psoriasis, erythroblastosis fetalis, eosinophilic faciitis, Shulman's syndrome, Felty's syndrome, filariasis, cyclitis, chronic cyclitis, heterochronic cyclitis, Fuch's cyclitis, IgA nephropathy, Henoch-Schonlein purpura, graft versus host disease, transplantation rejection, cardiomyopathy, Eaton-Lambert syndrome, relapsing polychondritis, cryoglobulinemia, Waldenstrom's macroglobulemia, Evan's syndrome, acute respiratory distress syndrome, pulmonary inflammation, osteoporosis, delayed type hypersensitivity and autoimmune gonadal failure.

Accordingly, the methods described herein encompass treatment of disorders of B lymphocytes (e.g., systemic lupus erythematosus, Goodpasture's syndrome, rheumatoid arthritis, and type I diabetes), Th₁-lymphocytes (e.g., rheumatoid arthritis, multiple sclerosis, psoriasis, Sjorgren's syndrome, Hashimoto's thyroiditis, Grave's disease, primary biliary cirrhosis, Wegener's granulomatosis, tuberculosis, or graft versus host disease), or Th₂-lymphocytes (e.g., atopic dermatitis, systemic lupus erythematosus, atopic asthma, rhinoconjunctivitis, allergic rhinitis, Omenn's syndrome, systemic sclerosis, or chronic graft versus host disease). Generally, disorders involving dendritic cells involve disorders of Th₁-lymphocytes or Th₂-lymphocytes.

In some embodiments, the immunological disorder is a T cell-mediated immunological disorder, such as a T cell disorder in which activated T cells associated with the disorder express CD40. CD40 binding agent s can be administered to deplete such CD40-expressing activated T cells. In a specific embodiment, administration of CD40 antibodies or agents can deplete CD40-expressing activated T cells, while resting T cells are not substantially depleted by the CD40 or agent. In this context, “not substantially depleted” means that less than about 60%, or less than about 70% or less than about 80% of resting T cells are not depleted.

The CD40 binding agents as described herein are also useful for treating or preventing a CD40-expressing cancer. Treatment or prevention of a CD40-expressing cancer, according to the methods described herein, is achieved by administering to a patient in need of such treatment or prevention an effective amount of the CD40 binding agent, whereby the agent (i) binds to CD40-expressing cancer cells and (ii) exerts a cytotoxic or cytostatic effect to deplete or inhibit the proliferation of the CD40-expressing cancer cells.

CD40-expressing cancers that can be treated or prevented by the methods described herein include, for example, leukemia, such as acute leukemia, acute lymphocytic leukemia, acute myelocytic leukemia (e.g., myeloblastic, promyelocytic, myelomonocytic, monocytic, or erythroleukemia), chronic leukemia, chronic myelocytic (granulocytic) leukemia, or chronic lymphocytic leukemia; Polycythemia vera; Lymphoma (e.g., Hodgkin's disease or Non-Hodgkin's disease); multiple myeloma, Waldenstrom's macroglobulinemia; heavy chain disease; solid tumors such sarcomas and carcinomas (e.g., fibrosarcoma, myxosarcoma, liposarcoma, chondrosarcoma, osteogenic sarcoma, osteosarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular tumor, lung carcinoma, small cell lung carcinoma, non small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, retinoblastoma, nasopharyngeal carcinoma, or esophageal carcinoma).

CD40 expressing types of non-Hodgkin's lymphoma include indolent (e.g., follicular, marginal zone, small cell lymphocytic lymphoma), aggressive (e.g., mantle cell) and highly aggressive (e.g., Burkitt's lymphoma, acute lymphoblastic leukemia) and diffuse large B-cell lymphoma (e.g., germinal center (e.g., BCL-6 positive) or activated B-cell lymphoma). In some embodiments, the non-Hodgkin's lymphoma is a highly aggressive lymphoma, such as Burkitt's lymphoma, acute lymphoblastic leukemia) or diffuse large B-cell lymphoma.

In some embodiments, the patient has a progressive (i.e., not stable), relapsed or refractory disease.

### Pharmaceutical Compositions and Administration Thereof

A composition comprising a CD40 binding agent (e.g., a CD40 antibody) can be administered to a patient having or at risk of having an immunological disorder or a CD40-expressing cancer. The invention further provides for the use of a CD40 binding agent (e.g., a CD40 antibody) in the manufacture of a medicament for prevention or treatment of a CD40 expressing cancer or immunological disorder. The term “patient” as used herein means any mammalian patient to which a CD40-binding agent can be administered, including, e.g., humans and non-human mammals, such as primates, rodents, and dogs. Patients specifically intended for treatment using the methods described herein include humans. The antibodies or agents can be administered either alone or in combination with other compositions in the prevention or treatment of the immunological disorder or CD40-expressing cancer.

Various delivery systems are known and can be used to administer the CD40 binding agent. Methods of introduction include but are not limited to intradermal, intramuscular, intraperitoneal, intravenous, subcutaneous, intranasal, epidural, and oral routes. The CD40 binding agent can be administered, for example by influsion, bolus or injection, and can be administered together with other biologically active agents such as chemotherapeutic agents. Administration can be systemic or local:

In specific embodiments, the CD40 binding agent composition is administered by injection, by means of a catheter, by means of a suppository, or by means of an implant, the implant being of a porous, non-porous, or gelatinous material, including a membrane, such as a sialastic membrane, or a fiber. Typically, when administering the composition, materials to which the CD40 antibody or agent does not absorb are used.

In other embodiments, the CD40 antibody or agent is delivered in a controlled release system. In one embodiment, a pump may be used (see, e.g., Langer, 1990, Science 249:1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228:190; During et al., 1989, Ann. Neurol. 25:351; Howard et al., 1989, J. Neurosurg. 71:105.) Other controlled release systems are discussed, for example, in Langer, supra.

A CD40 binding agent (e.g., a CD40 antibody) can be administered as pharmaceutical compositions comprising a therapeutically effective amount of the binding agent and one or more pharmaceutically compatible ingredients. For example, the pharmaceutical composition typically includes one or more pharmaceutical carriers (e.g., sterile liquids, such as water and oils, including those of petroleum, animal, vegetable or synthetic origin, such as peanut oil, soybean oil, mineral oil, sesame oil and the like). Water is a more typical carrier when the pharmaceutical composition is administered intravenously. Saline solutions and aqueous dextrose and glycerol solutions can also be employed as liquid carriers, particularly for injectable solutions. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. Other suitable pharmaceutical excipients include amino acids (e.g., arginine, histidine, glycine), surfactants (e.g., polysorbates) and sugars and sugar alcohols (e.g., sucrose or sorbitol and other polyols (e.g., trehalose)). The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulations can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E.W. Martin. Such compositions will contain a therapeutically effective amount of the nucleic acid or protein, typically in purified form, together with a suitable amount of carrier so as to provide the form for proper administration to the patient. The formulations correspond to the mode of administration.

In typical embodiments, the pharmaceutical composition is formulated in accordance with routine procedures as a pharmaceutical composition adapted for intravenous administration to human beings. Typically, compositions for intravenous administration are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical is administered by injection, an ampoule of sterile water for injection or saline can be provided so that the ingredients can be mixed prior to administration.

Further, the pharmaceutical composition can be provided as a pharmaceutical kit comprising (a) a container containing a CD40 binding agent (e.g., a CD40 antibody) in lyophilized form and (b) a second container containing a pharmaceutically acceptable diluent (e.g., sterile water) for injection. The pharmaceutically acceptable diluent can be used for reconstitution or dilution of the lyophilized CD40 antibody or agent. Optionally associated with such container(s) can be a notice in the form prescribed by a governmental agency regulating the manufacture, use or sale of pharmaceuticals or biological products, which notice reflects approval by the agency of manufacture, use or sale for human administration.

In some embodiments, the pharmaceutical compositions comprising the CD40 binding agent can further comprise a therapeutic agent, either conjugated or unconjugated to the binding agent. The CD40 antibody or CD40 binding agent can be co-administered in combination with one or more therapeutic agents for the treatment or prevention of immunological disorders or CD40-expressing cancers. For example, combination therapy can include a cytostatic, cytotoxic, or immunomodulatory agent. Combination therapy can also include, e.g., administration of an agent that targets a receptor or receptor complex other than CD40 on the surface of activated lymphocytes, dendritic cells or CD40-expressing cancer cells. An example of such an agent includes a second, non-CD40 antibody that binds to a molecule at the surface of an activated lymphocyte, dendritic cell or CD40-expressing cancer cell. Another example includes a ligand that targets such a receptor or receptor complex. Typically, such an antibody or ligand binds to a cell surface receptor on activated lymphocytes, dendritic cell or CD40-expressing cancer cell and enhances the cytotoxic or cytostatic effect of the CD40 antibody by delivering a cytostatic or cytotoxic signal to the activated lymphocyte, dendritic cell or CD40-expressing cancer cell.

Such combinatorial administration can have an additive or synergistic effect on disease parameters (e.g., severity of a symptom, the number of symptoms, or frequency of relapse).

With respect to therapeutic regimens for combinatorial administration, in a specific embodiment, a CD40 antibody or CD40 binding agent is administered concurrently with a therapeutic agent. In another specific embodiment, the therapeutic agent is administered prior or subsequent to administration of the CD40 antibody or CD40 binding agent, by at least an hour and up to several months, for example at least an hour, five hours, 12 hours, a day, a week, a month, or three months, prior or subsequent to administration of the CD40 antibody or CD40 binding agent.

Useful classes of cytotoxic or immunomodulatory agents include, for example, antitubulin agents, auristatins (e.g., MMAE, or MMA), DNA minor groove binders, DNA replication inhibitors, alkylating agents (e.g., platinum complexes such as cis-platin, mono(platinum), bis(platinum) and tri-nuclear platinum complexes and carboplatin), anthracyclines, antibiotics, antifolates, antimetabolites, chemotherapy sensitizers, duocarmycins, etoposides, fluorinated pyrimidines, ionophores, lexitropsins, nitrosoureas, platinols, pre-forming compounds, purine antimetabolites, puromycins, radiation sensitizers, steroids, taxanes, topoisomerase inhibitors, vinca alkaloids, or the like.

Individual cytotoxic or immunomodulatory agents include, for example, an androgen, anthramycin (AMC), asparaginase, 5-azacytidine, azathioprine, bleomycin, bortezomib (e.g., VELCADE), busulfan, buthionine sulfoximine, camptothecin, carboplatin, carmustine (BSNU), CC-1065, chlorambucil, cisplatin, colchicine, cyclophosphamide, cytarabine, cytidine arabinoside, cytochalasin B, dacarbazine, dactinomycin (formerly actinomycin), daunorubicin, decarbazine, docetaxel, doxorubicin, an estrogen, 5-fluordeoxyuridine, 5-fluorouracil, gramicidin D, hydroxyurea, idarubicin, ifosfamide, irinotecan, lenalidomide (REVLIMID), lomustine (CCNU), mechlorethamine, melphalan, 6-mercaptopurine, methotrexate, mithramycin, mitomycin C, mitoxantrone, nitroimidazole, paclitaxel, plicamycin, procarbizine, streptozotocin, tenoposide, 6-thioguanine, thioTEPA, topotecan, vinblastine, vincristine, vinorelbine, VP-16 and VM-26.

In some typical embodiments, the therapeutic agent is a cytotoxic agent. Suitable cytotoxic agents include, for example, dolastatins (e.g., auristatin E, AFP, MMAF, MMAE, AEB or AEVB), DNA minor groove binders (e.g., enediynes and lexitropsins), duocarmycins, taxanes (e.g., paclitaxel and docetaxel), puromycins, vinca alkaloids, CC-1065, SN-38, topotecan, morpholino-doxorubicin, rhizoxin, cyanomorpholino-doxorubicin, echinomycin, combretastatin, netropsin, epothilone A and B, estramustine, cryptophysins, cemadotin, maytansinoids, discodermolide, eleutherobin, or mitoxantrone.

In some embodiments, the cytotoxic agent is a conventional chemotherapeutic such as, for example, doxorubicin, paclitaxel, melphalan, vinca alkaloids, methotrexate, mitomycin C or etoposide. In addition, potent agents such as CC-1065 analogues, calicheamicin, maytansine, analogues of dolastatin 10, rhizoxin, and palytoxin can be linked to the CD40 antibodies or agents thereof.

In specific embodiments, the cytotoxic or cytostatic agent is auristatin E (also known in the art as dolastatin-10) or a derivative thereof. Typically, the auristatin E derivative is, e.g., an ester formed between auristatin E and a keto acid. For example, auristatin E can be reacted with paraacetyl benzoic acid or benzoylvaleric acid to produce AEB and AEVB, respectively. Other typical auristatin derivatives include AFP, MMAF, and MMAE. The synthesis and structure of auristatin E and its derivatives are described in, for example, U.S. Patent Application Publication Nos. 2004-0157782 A1 and 2005-0238649; International Patent Application No. PCT/US03/24209, International Patent Application No. PCT/US02/13435, and U.S. Pat. Nos. 6,884,869; 6,323,315; 6,239,104; 6,034,065; 5,780,588; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4,879,278; 4,816,444; and 4,486,414; the disclosures of which are incorporated by reference herein.

In specific embodiments, the cytotoxic agent is a DNA minor groove binding agent. (See, e.g., U.S. Pat. No. 6,130,237.) For example, in some embodiments, the minor groove binding agent is a CBI compound. In other embodiments, the minor groove binding agent is an enediyne (e.g., calicheamicin).

Examples of anti-tubulin agents include, but are not limited to, taxanes (e.g., Taxol® (paclitaxel), Taxotere® (docetaxel)), T67 (Tularik), vinca alkyloids (e.g., vincristine, vinblastine, vindesine, and vinorelbine), and dolastatins (e.g., auristatin E, AFP, MMAF, MMAE, AEB, AEVB). Other antitubulin agents include, for example, baccatin derivatives, taxane analogs (e.g., epothilone A and B), nocodazole, colchicine and colcimid, estramustine, cryptophysins, cemadotin, maytansinoids, combretastatins, discodermolide, and eleutherobin.

In some embodiments, the cytotoxic agent is a maytansinoid, another group of anti-tubulin agents. For example, in specific embodiments, the maytansinoid is maytansine or DM-1 (InimunoGen, Inc.; see also Chari et al., 1992, Cancer Res. 52:127-131).

In some embodiments, the therapeutic agent is not a radioisotope.

In some embodiments, the cytotoxic or immunomodulatory agent is an antimetabolite. The antimetabolite can be, for example, a purine antagonist (e.g., azothioprine or mycophenolate mofetil), a dihydrofolate reductase inhibitor (e.g., methotrexate), acyclovir, gangcyclovir, zidovudine, vidarabine, ribavarin, azidothymidine, cytidine arabinoside, amantadine, dideoxyuridine, iododeoxyuridine, poscarnet, or trifluridine.

In other embodiments, the cytotoxic or immunomodulatory agent is tacrolimus, cyclosporine or rapamycin. In further embodiments, the cytotoxic agent is aldesleukin, alemtuzumab, alitretinoin, allopurinol, altretamine, amifostine, anastrozole, arsenic trioxide (e.g., TRISENOX), bexarotene, bexarotene, calusterone, capecitabine, celecoxib, cladribine, Denileukin diflitox, dexrazoxane, dromostanolone propionate, epirubicin, estramustine, exemestane, Filgrastim, floxuridine, fludarabine, fulvestrant, gemcitabine, gemtuzumab ozogamicin, goserelin, idarubicin, ifosfamide, imatinib mesylate, Interferon alfa-2a, irinotecan, letrozole, leucovorin, levamisole, meclorethamine or nitrogen mustard, megestrol, mesna, methotrexate, methoxsalen, mitomycin C, mitotane, nandrolone phenpropionate, oprelvekin, oxaliplatin, pamidronate, pegademase, pegaspargase, pegfilgrastim, pentostatin, pipobroman, plicamycin, porfimer sodium, procarbazine, quinacrine, rasburicase, revlimid, Sargramostim, streptozocin, tamoxifen, temozolomide, teniposide, testolactone, thioguanine, toremifene, Tositumomab, Trastuzumab, tretinoin, uracil mustard, valrubicin, vinblastine, vincristine, vinorelbine and zoledronate.

In additional embodiments, the drug is a humanized anti-HER2 monoclonal antibody; RITUXAN (rituximab; Genentech, Inc., South San Francisco, Calif.); a chimeric anti-CD20 monoclonal antibody); OVAREX (AltaRex Corporation, MA); PANOREX (Glaxo Wellcome, NC; a murine IgG2a antibody); Cetuximab Erbitux (Inclone Systems Inc., NY; an anti-EGFR IgG chimeric antibody); Vitaxin (MedImmune, Inc., MD); Campath I/H (Leukosite, MA; a humanized IgG1 antibody); Smart M195 (Protein Design Labs, Inc., CA; a humanized anti-CD33 IgG antibody); LymphoCide (Immunomedics, Inc., NJ; a humanized anti-CD22 IgG antibody); Smart ID10 (Protein Design Labs, Inc., CA; a humanized anti-HLA-DR antibody); Oncolym (Techniclone, Inc., CA; a radiolabeled murine anti-HLA-Dr10 antibody); Allomune (BioTransplant, CA; a humanized anti-CD2 mAb); Avastin (Genentech, Inc., CA; an anti-VEGF humanized antibody); Epratuzamab (Immunomedics, Inc., NJ and Amgen, CA; an anti-CD22 antibody); and CEAcide (Immunomedics, NJ; a humanized anti-CEA antibody).

Other suitable antibodies include, but are not limited to, antibodies against the following antigens: CA125, CA15-3, CA19-9, L6, Lewis Y, Lewis X, alpha fetoprotein, CA 242, placental alkaline phosphatase, prostate specific antigen, prostatic acid phosphatase, epidermal growth factor, MAGE-1, MAGE-2, MAGE-3, MAGE-4, anti transferrin receptor, p97, MUC1-KLH, CEA, gp100, MART1, Prostate Specific Antigen, IL-2 receptor, CD20, CD52, CD33, CD22, human chorionic gonadotropin, CD38, mucin, P21, MPG, and Neu oncogene product.

In some embodiments, the therapeutic agent is an immunomodulatory agent. The immunomodulatory agent can be, for example, gancyclovir, etanercept, tacrolimus, cyclosporine, raparnycin, cyclophosphamide, azathioprine, mycophenolate mofetil or methotrexate. Alternatively, the immunomodulatory agent can be, for example, a glucocorticoid (e.g., cortisol or aldosterone) or a glucocorticoid analogue (e.g., dexamethasone, prednisone or methyl-prednisolone (e.g., SOLU-MEDROL)).

Suitable cyclooxygenase inhibitors include meclofenamic acid, mefenamic acid, carprofen, diclofenac, diflunisal, fenbufen, fenoprofen, ibuprofen, indomethacin, ketoprofen, nabumetone, naproxen, sulindac, tenoxicam, tolmetin, and acetylsalicylic acid.

Suitable lipoxygenase inhibitors include redox inhibitors (e.g., catechol butane derivatives, nordihydroguaiaretic acid (NDGA), masoprocol, phenidone, Ianopalen, indazolinones, naphazatrom, benzofuranol, alkylhydroxylamine), and non-redox inhibitors (e.g., hydroxythiazoles, methoxyalkylthiazoles, benzopyrans and derivatives thereof, methoxytetrahydropyran, boswellic acids and acetylated derivatives of boswellic acids, and quinolinemethoxyphenylacetic acids substituted with cycloalkyl radicals), and precursors of redox inhibitors.

Other suitable lipoxygenase inhibitors include antioxidants (e.g., phenols, propyl gallate, flavonoids and/or naturally occurring substrates containing flavonoids, hydroxylated derivatives of the flavones, flavonol, dihydroquercetin, luteolin, galangin, orobol, derivatives of chalcone, 4,2′,4′-trihydroxychalcone, ortho-aminophenols, N-hydroxyureas, benzofuranols, ebselen and species that increase the activity of the reducing selenoenzymes), iron chelating agents (e.g., hydroxamic acids and derivatives thereof, N-hydroxyureas, 2-benzyl-1-naphthol, catechols, hydroxylamines, carnosol trolox C, catechol, naphthol, sulfasalazine, zyleuton, 5-hydroxyanthranilic acid and 4-(omega-arylalkyl)phenylalkanoic acids), imidazole-containing compounds (e.g., ketoconazole and itraconazole), phenothiazines, and benzopyran derivatives.

Yet other suitable lipoxygenase inhibitors include inhibitors of eicosanoids (e.g., octadecatetraenoic, eicosatetraenoic, docosapentaenoic, eicosahexaenoic and docosahexaenoic acids and esters thereof, PGE1 (prostaglandin E1), PGA2 (prostaglandin A2), viprostol, 15-monohydroxyeicosatetraenoic, 15-monohydroxy-eicosatrienoic and 15-monohydroxyeicosapentaenoic acids, and leukotrienes B5, C5 and D5), compounds interfering with calcium flows, phenothiazines, diphenylbutylamines, verapamil, fuscoside, curcurnin, chlorogenic acid, caffeic acid, 5,8,11,14-eicosatetrayenoic acid (ETYA), hydroxyphenylretinamide, Ionapalen, esculin, diethylcarbamazine, phenantroline, baicalein, proxicromil, thioethers, diallyl sulfide and di-(1-propenyl)sulfide.

Leukotriene receptor antagonists include calcitriol, ontazolast, Bayer Bay-x-1005, Ciba-Geigy CGS-25019C, ebselen, Leo Denmark ETH-615, Lilly LY-293111, Ono ONO-4057, Terumo TMK-688, Boehringer Ingleheim BI-RM-270, Lilly LY 213024, Lilly LY 264086, Lilly LY 292728, Ono ONO LB457, Pfizer 105696, Perdue Frederick PF 10042, Rhone-Poulenc Rorer RP 66153, SmithKline Beecham SB-201146, SmithKline Beecharn SB-201993, SmithKline Beecham SB-209247, Searle SC-53228, Sumitamo SM 15178, American Home Products WAY 121006, Bayer Bay-o-8276, Warner-Lambert CI-987, Warner-Lambert CI-987BPC-1SLY 223982, Lilly LY 233569, Lilly LY-255283, MacroNex MNX-160, Merck and Co. MK-591, Merck and Co. MK-886, Ono ONO-LB-448, Purdue Frederick PF-5901, Rhone-Poulenc Rorer RG 14893, Rhone-Poulenc Rorer RP 66364, Rhone-Poulenc Rorer RP 69698, Shionoogi S-2474, Searle SC-41930, Searle SC-50505, Searle SC-51146, Searle SC-52798, SmithKline Beecham SKand F-104493, Leo Denmark SR-2566, Tanabe T-757 and Teijin TEI-1338.

In some embodiments, the therapeutic agent is not vitamin C or cycloheximide. In some embodiments, the therapeutic agent is IMiD3 [3-(4-amino-1-oxo-1,3-dihydro-isoindol-2-yl)-piperidine-2,6-dione]. In other embodiments, the therapeutic agent is not IMiD3. In some embodiments, the therapeutic agent is not CD40L. In some embodiments, the therapeutic agent is not thalidomide. In some embodiments, the therapeutic agent is not an exogenous cytokine. In a specific embodiment, the therapeutic agent is not IL-4.

In some embodiments, the therapy is a combined therapy, such as CHOP (cyclophosphamide, doxorubicin, vincristine, prednisolone), R-CHOP (rituximab, cyclophosphamide, doxorubicin, vincristine, prednisolone), ACVBP (doxorubicin, cyclophosphamide, vindesine, bleomycin, prednisone), ICE (Idarubicin, high dose Cytosine arabinoside, Etoposide), R—ICE (rituximab, Idarubicin, high dose Cytosine arabinoside, Etoposide), CNOP (cyclophosphamide, mitoxantrone, vincristine, prednisone), m-BACOD (methotrexate, bleomycin, doxorubicin, cyclophosphamide, vincristine, dexametbasone, leucovorin), MACOP-B (methotrexate, doxorubicin, cyclophosphamide, vincristine, prednisone, bleomycin, leucovorin), ProMACE CytaBOM (prednisone, doxorubici, cyclophosphamide, etoposide, cytarabine, bleomycin, vincristine, methotrexate, leucovorin) or GVD (gemcitabine, vincristine, doxorubicin).

In some embodiments, a patient having multiple myeloma is treated with a CD40 binding agent and lenalidomide. The patient may have, for example, progressive or refractory multiple myeloma. The patient may receive, for example, from one to about four weekly doses (e.g., from about 0.5 mg/kg to about 16 mg/kg) of the CD40 binding agent.

In some embodiments, a patient having a non-Hodgkin's lymphoma (NHL) is treated with a CD40 binding agent (e.g., from about 0.5 mg/kg to about 8 mg/kg) and CHOP or R-CHOP. The patient can be newly diagnosed (e.g., previously untreated for NHL), or previously treated (e.g., either relapsed or having failed a prior treatment). The non-Hodgkin's lymphoma can be indolent or aggressive.

In some embodiments, a patient having a non-Hodgkin's lymphoma (NHL) is treated with a CD40 binding agent (e.g., from about 0.5 mg/kg to about 8 mg/kg) and ICE or R—ICE. The patient can be newly diagnosed (e.g., previously untreated for NHL), or previously treated (e.g., either relapsed or having failed a prior treatment). The non-Hodgkin's lymphoma can be indolent or aggressive.

In some embodiments, a patient having diffuse large B cell lymphoma (DLBCL) is treated with a CD40 binding agent and R-CHOP or CHOP. The patient can be newly diagnosed (e.g., previously untreated for DLBCL), or previously treated (e.g., either relapsed or having failed a prior treatment). In some embodiments, CHOP is administered without rituximab.

In some embodiments, a patient having mantle cell lymphoma or Waldenstroms' macroglobulinema is treated with a CD40 binding agent and rituximab. In some embodiments, a patient having chronic lymphocytic leukemia (CLL) is treated with a CD40 binding agent and rituximab.

### Articles of Manufacture

In another aspect, an article of manufacture containing materials useful for the treatment of the disorders described above is included. The article of manufacture comprises a container and a label. Suitable containers include, for example, bottles, vials, syringes, and test tubes. The containers may be formed from a variety of materials such as glass or plastic. The container holds a composition that is effective for treating the condition and may have a sterile access port. For example, the container may be an intravenous solution bag or a vial having a stopper pierceable by a hypodermic injection needle. The active agent in the composition is the CD40 binding agent. The label on or associated with the container indicates that the composition is used for treating the condition of choice. The article of manufacture may further comprise a second container comprising a pharmaceutically-acceptable buffer, such as phosphate-buffered saline, Ringer's solution, and dextrose solution. It may further include other materials desirable from a commercial and user standpoint, including other buffers, diluents, filters, needles, syringes, and package inserts with instructions for use.

### ATCC Deposit

An ATCC deposit of monoclonal antibody S2C6 was made on May 25, 1999 pursuant to the terms of the Budapest Treaty on the international recognition of the deposit of microorganisms for purposes of patent procedure. The ATCC is located at University Boulevard, Manassas, Va. 20110-2209, USA. This ATCC deposit was given an accession number of PTA-110. The ATCC is located at 10801 University Boulevard, Manassas, Va. 20110-2209, USA. Any deposit is provided as a convenience to those of skill in the art and is not an admission that a deposit is required under 35 U.S.C. Section 112. That described herein is not to be limited in scope by the antibody deposited, since the deposited embodiment is intended as a single illustration of certain aspects of the invention and any antibody that is functionally equivalent is within the scope of this invention. The deposit of material herein does not constitute an admission that the written description herein contained is inadequate to enable the practice of any aspect of the invention, including the best mode thereof, nor is it to be construed as limiting the scope of the claims to the specific illustrations that it represents. Indeed, various modifications of the invention in addition to those shown and described herein will become apparent to those skilled in the art from the foregoing description and fall within the scope of the appended claims.

The invention is further described in the following examples, which are not intended to limit the scope of the invention. Cell lines described in the following examples were maintained in culture according to the conditions specified by the American Type Culture Collection (ATCC) or Deutsche SammIung von Mikroorganismen und Zelilkulturen GmbH, Braunschweig, Germany (DMSZ). Cell culture reagents were obtained from Invitrogen Corp. (Carlsbad, Calif.).

### EXAMPLES

### Example 1

### Production of Humanized CD40 Antibody

A humanized CD40 antibody was constructed generally by importing the CDRs of the murine CD40 donor antibody into a recipient human antibody. The donor antibody was the murine monoclonal antibody S2C6, described in U.S. Pat. No. 6,838,261, and demonstrated to provide strong, growth-promoting signals to B-lymphocytes. See, e.g., Paulie et. al., 2000, J. Immunol. 142:590. Consensus sequences for the human subgroup III heavy chain variable domain (SEQ ID NO:2) and for the human kappa subgroup I light chain variable domain (SEQ ID NO:13) were obtained, as generally described in Carter et al., 1992, Proc. Natl. Acad. Sci. USA 89:4285; U.S. Pat. No. 6,037,454, and U.S. Pat. No. 6,054,297 to use as the human recipient heavy and light chain domains. The humanized antibody variants were prepared as described in International Publication No. WO 2006/128103 (the disclosure of which is incorporated by reference herein). The sequences of the antibody variants is shown in the following Table 1 and 2.

TABLE 1
Heavy Chain Variable Domain

Ab/SEQ ID NO

| | |
|---|---|
| | 10 20 30 40 50 |
| sgn-0/3 | EVQLVESGGG LVQPGGSLRL SCAASGYSFT GYYIHWVRQA PGKGLEWVAR |
| sgn-1/4 | EVQLVESGGG LVQPGGSLRL SCAASGYSFT GYYIHWVRQA PGKGLEWVAR |
| sgn-2/5 | EVQLVESGGG LVQPGGSLRL SCAASGYSFT GYYIHWVRQA PGKGLEWVAR |
| sgn-4/6 | EVQLVESGGG LVQPGGSLRL SCAASGYSFT GYYIHWVRQA PGKGLEWVAR |
| sgn-14/17 | EVQLVESGGG LVQPGGSLRL SCAASGYSFT GYYIHWVRQA PGKGLEWVAR |
| sgn-15/8 | EVQLVESGGG LVQPGGSLRL SCAASGYSFT GYYIHWVRQA PGKGLEWVAR |
| sgn-16/9 | EVQLVESGGG LVQPGGSLRL SCAASGYSFT GYYIHWVRQA PGKGLEWVAR |
| sgn-17/6 | EVQLVESGGG LVQPGGSLRL SCAASGYSFT GYYIHWVRQA PGKGLEWVAR |
| sgn-18/6 | EVQLVESGGG LVQPGGSLRL SCAASGYSFT GYYIHWVRQA PGKGLEWVAR |
| sgn-19/7 | EVQLVESGGG LVQPGGSLRL SCAASGYSFT GYYIHWVRQA PGKGLEWVAR |
| sgn-22/10 | EVQLVESGGG LVQPGGSLRL SCAASGYSFT GYYIHWVRQA PGKGLEWVAR |
| sgn-23/11 | EVQLVESGGG LVQPGGSLRL SCAASGYSFT GYYIHWVRQA PGKGLEWVAR |
| sgn-26/10 | EVQLVESGGG LVQPGGSLRL SCAASGYSFT GYYIHWVRQA PGKGLEWVAR |
| sgn-27/11 | EVQLVESGGG LVQPGGSLRL SCAASGYSFT GYYIHWVRQA PGKGLEWVAR |
| | CDR-H1 |
| | 60 70 80 90 100 |
| sgn-0/3 | VIPNNGGTSY NQKFKGRFTI SRDNSKNTLY LQMNSLRAED TAVYYCAREG |
| sgn-1/4 | VIPNNGGTSY NQKFKGRFTI SVDNSKNTLY LQMNSLRAED TAVYYCAREG |
| sgn-2/5 | VIPNNGGTSY NQKFKGRFTI SRDKSKNTLY LQMNSLRAED TAVYYCAREG |
| sgn-4/6 | VIPNNGGTSY NQKFKGRATL SVDNSKNTAY LQNNSLRAED TAVYYCAREG |
| sgn-14/7 | VIPNNGGTSY NQKFKGRFTL SVDNSKNTAY LQMNSLRAED TAVYYCAREG |
| sgn-15/8 | VIPNNGGTSY NQKFKGRATI SVDNSKNTAY LQMNSLRAED TAVYYCAREG |
| sgn-16/9 | VIPNNGGTSY NQKFKGRATL SVDNSKNTLY LQMNSLRAED TAVYYCAREG |
| sgn-17/6 | VIPNNGGTSY NQKFKGRATL SVDNSKNTAY LQMNSLRAED TAVYYCAREG |
| sgn-18/6 | VIPNNGGTSY NQKFKGRATL SVDNSKNTAY LQMNSLRAED TAVYYCAREG |
| sgn-19/7 | VIPNNGGTSY NQKFKGRFTL SVDNSKNTAY LQMNSLRAED TAVYYCAREG |
| sgn-22/10 | VIPNAGGTSY NQKFKGRFTL SVDNSKNTAY LQMNSLRAED TAVYYCAREG |
| sgn-23/11 | VIPNQGGTSY NQKFKGRFTL SVDNSKNTAY LQMNSLRAED TAVYYCAREG |
| sgn-26/10 | VIPNAGGTSY NQKFKGRFTL SVDNSKNTAY LQMNSLRAED TAVYYCAREG |
| sgn-27/11 | VIPNQGGTSY NQKFKGRFTL SVDNSKNTAY LQMNSLRAED TAVYYCAREG |
| | CDR-H2 |
| | 110 |
| sgn-0/3 | I--- YWWGQGTLV TVS |
| sgn-1/4 | I--- YWWGQGTLV TVS |
| sgn-2/5 | I--- YWWGQGTLV TVS |
| sgn-4/6 | I--- YWWGQGTLV TVS |
| sgn-14/7 | I--- YWWGQGTLV TVS |
| sgn-15/8 | I--- YWWGQGTLV TVS |
| sgn-16/9 | I--- YWWGQGTLV TVS |
| sgn-17/6 | I--- YWWGQGTLV TVS |
| sgn-18/6 | I--- YWWGQGTLV TVS |
| sgn-19/7 | I--- YWWGQGTLV TVS |
| sgn-22/10 | I--- YWWGQGTLV TVS |
| sgn-23/11 | I--- YWWGQGTLV TVS |
| sgn-26/10 | I--- YWWGQGTLV TVS |
| sgn-27/11 | I--- YWWGQGTLV TVS |
| | CDR-H3 |

TABLE 2
Light Chain Variable Domain

Ab/SEQ ID NO

| | |
|---|---|
| | 10 20 30 40 50 |
| sgn-0/14 | DIQMTQSPSS LSASVGDRVT ITCRSSQSLV HSNGNTFLHW YQQKPGKAPK |
| sgn-1/14 | DIQMTQSPSS LSASVGDRVT ITCRSSQSLV HSNGNTFLHW YQOKPGKAPK |
| sgn-2/14 | DIQMTQSPSS LSASVGDRVT ITCRSSQSLV HSNGNTFLHW YQQKPGKAPK |
| sgn-4/14 | DIQMTQSPSS LSASVGDRVT ITCRSSQSLV HSNGNTFLHW YQQKPGKAPK |
| sgn-14/14 | DIQMTQSPSS LSASVGDRVT ITCRSSQSLV HSNGNTFLHW YQQKPGKAPK |
| sgn-15/14 | DIQMTQSPSS LSASVGDRVT ITCRSSQSLV HSNGNTFLHW YQQKPGKAPK |
| sgn-16/14 | DIQMTQSPSS LSASVGDRVT ITCRSSQSLV HSNGNTFLHW YQQKPGKAPK |
| sgn-17/15 | DVQVTQSPSS LSASVGDRVT ITCRSSQSLV HSNGNTFLHW YQQKPGKAPK |
| sgn-18/16 | DIQMTQSPSS LSASVGDRVT ITCRSSQSLV HSNGNTFLHW YQQKPGKAPK |
| sgn-19/16 | DIQMTQSPSS LSASVGDRVT ITCRSSQSLV HSNGNTFLHW YQQKPGKAPK |
| sgn-22/14 | DIOMTQSPSS LSASVGDRVT ITCRSSQSLV HSNGNTFLHW YQQKPGKAPK |
| sgn-23/14 | DIQMTQSPSS LSASVGDRVT ITCRSSQSLV HSNGNTFLHW YQQKPGKAPK |
| sgn-26/16 | DIQMTQSPSS LSASVGDRVT ITCRSSQSLV HSNGNTFLHW YQQKPGKAPK |
| sgn-27/16 | DIQMTQSPSS LSASVGDRVT ITCRSSQSLV HSNGNTFLHW YQQKPGKAPK |
| | CDR-L1 |
| | 60 70 80 90 100 |
| sgn-0/14 | LLIYTVSNRF SGVPSRFSGS GSGTDFTLTI SSLQPEDFAT YYCSQTTHVP |
| sgn-1/14 | LLIYTVSNRF SGVPSRFSGS GSGTDFTLTI SSLQPEDFAT YYCSQTTHVP |
| sgn-2/14 | LLIYTVSNRF SGVPSRFSGS GSGTDFTLTI SSLQPEDFAT YYCSQTTHVP |
| sgn-4/14 | LLIYTVSNRF SGVPSRFSGS GSGTDFTLTI SSLQPEDFAT YYCSQTTHVP |
| sgn-14/14 | LLIYTVSNRF SGVPSRFSGS GSGTDFTLTI SSLQPEDFAT YYCSQTTHVP |
| sgn-15/14 | LLIYTVSNRF SGVPSRFSGS GSGTDFTLTI SSLQPEDFAT YYCSQTTHVP |
| sgn-16/14 | LLIYTVSNRF SGVPSRFSGS GSGTDFTLTI SSLQPEDFAT YYCSQTTHVP |
| sgn-17/15 | LLIYTVSNRF SGVPSRFSGS GSGTDFTLTI SSLQPEDFAT YYCSQTTHVP |
| sgn-18/16 | LLIYTVSNRF SGVPSRFSGS GSGTDFTLTI SSLQPEDFAT YFCSQTTHVP |
| sgn-19/16 | LLIYTVSNRF SGVPSRFSGS GSGTDFTLTI SSLQPEDFAT YFCSQTTHVP |
| sgn-22/14 | LLIYTVSNRF SGVPSRFSGS GSGTDFTLTI SSLQPEDFAT YYCSQTTHVP |
| sgn-23/14 | LLIYTVSNRF SGVPSRFSGS GSGTDFTLTI SSLQPEDFAT YYCSQTTHVP |
| sgn-26/16 | LLIYTVSNRF SGVPSRFSGS GSGTDFTLTI SSLQPEDFAT YFCSQTTHVP |
| sgn-27/16 | LLIYTVSNRF SGVPSRFSGS GSGTDFTLTI SSLQPEDFAT YFCSQTTHVP |
| | CDR-L2 CDR-L3 |
| | 110 |
| sgn-0/14 | WTFGQGTKVE IKR |
| sgn-1/14 | WTFGQGTKVE IKR |
| sgn-2/14 | WTFGQGTKVE IKR |
| sgn-4/14 | WTFGQGTKVE IKR |
| sgn-14/14 | WTFGQGTKVE IKR |
| sgn-15/14 | WTFGQGTKVE IKR |
| sgn-16/14 | WTFGQGTKVE IKR |
| sgn-17/15 | WTFGQGTKVE IKR |
| sgn-18/16 | WTFGQGTKVE IKR |
| sgn-19/16 | WTFGQGTKVE IKR |
| sgn-22/14 | WTFGQGTKVE IKR |
| sgn-23/14 | WTFGQGTKVE IKR |
| sgn-26/16 | WTFGQGTKVE IKR |
| sgn-27/16 | WTFGQGTKVE IKR |

Antibodies constructed using these variant light and heavy chain variable domains were analyzed for binding activity. Each antibody was diluted to equivalent concentrations, and then serially diluted. The diluted antibodies were assayed for binding to CD40 immobilized on microassay plates. Affinity binding data for the variant antibodies are shown below in Table 3. Antibodies showing binding activity approaching that of the parent murine antibody were sgn-14, sgn-18, sgn-19, sgn-22, sgn-23, sgn-26, and sgn-27, with variants sgn-14, sgn-18, sgn-26, and sgn-27 more closely approaching that of the parent murine antibody, SGN-14, and variant sgn-26 showing the best performance in these assays.

TABLE 3

| | Heavy Chain | Light Chain | Binding | Binding | Binding |
|---|---|---|---|---|---|
| Antibody | Variable Domain | Variable Domain | Data 1 | Data 2 | Data 3 |
| SGN-14 | SEQ ID NO: 1 | SEQ ID NO: 12 | 1.00 | 1.33 | 1.16 |
| | Donor | Donor | | | |
| hu sgn-0 | SEQ ID NO: 3 | SEQ ID NO: 14 | 75.31 | 75.31 | — |
| | Template | Template | | | |
| hu sgn-1 | SEQ ID NO: 4 | SEQ ID NO: 14 | 23.63 | 19.31 | — |
| | R72V | Template | | | |
| hu sgn-2 | SEQ ID NO: 5 | SEQ ID NO: 14 | 471.37 | 370.87 | — |
| | N74K | Template | | | |
| hu sgn-4 | SEQ ID NO: 6 | SEQ ID NO: 14 | 2.23 | 2.46 | — |
| | F68A I70L R72V L79A | Template | 1.83 | | |
| | | | 3.12 | | |
| | | | 1.41 | | |
| hu sgn-14 | SEQ ID NO: 7 | SEQ ID NO: 14 | 1/11 | 0.69 | — |
| | I70L R72V L79A | Template | 0.86 | 0.77 | |
| | | | 0.77 | | |
| hu sgn-15 | SEQ ID NO: 8 | SEQ ID NO: 14 | 10.54 | 2.71 | — |
| | F68A R72V L79A | Template | 4.67 | | |
| hu sgn-16 | SEQ ID NO: 9 | SEQ ID NO: 14 | 44.00 | 1.82 | — |
| | F68A I69L R72V | Template | 9.78 | 1.83 | |
| | | | 7.99 | | |
| hu sgn-17 | SEQ ID NO: 6 | SEQ ID NO: 15 | 3.60 | 3.56 | — |
| | F68A I70L R72V L79A | 12V M4V | 1.76 | | |
| hu sgn-18 | SEQ ID NO: 6 | SEQ ID NO: 16 | 0.96 | 1.03 | |
| | F68A I70L R72V L79A | Y92F | 0.67 | | |
| hu sgn-19 | SEQ ID NO: 7 | SEQ ID NO: 16 | 0.481 | 0.501 | — |
| | I70L R72V L79A | Y92F | 1.06 | 0.98 | |
| | | | | 0.92 | |
| | | | | 1.14 | |
| hu sgn-22 | SEQ ID NO: 10 | SEQ ID NO: 14 | 1.44 | 0.84 | — |
| | N55A I70L R72V L79A | Template | 1.41 | | |
| | | | 1.80 | | |
| | | | 0.93 | | |
| hu sgn-23 | SEQ ID NO: 11 | SEQ ID NO: 14 | 2.11 | 0.90 | — |
| | N55Q I70L R72V L79A | Template | 1.58 | | |
| | | | 2.38 | | |
| | | | 0.72 | | |
| hu sgn-26 | SEQ ID NO: 10 | SEQ ID NO: 16 | 0.92 | 0.92 | — |
| | N55A I70L R72V L79A | Y92F | 1.02 | | |
| | | | 1.02 | | |
| | | | 1.06 | | |
| hu sgn-27 | SEQ ID NO: 11 | SEQ ID NO: 16 | 1.04 | 1.40 | — |
| | N55Q I70L R72V L79A | Y92F | 1.03 | | |
| | | | 0.94 | | |
| | | | 0.83 | | |

### Example 2

### In Vitro Signaling by a Humanized CD40 Antibody

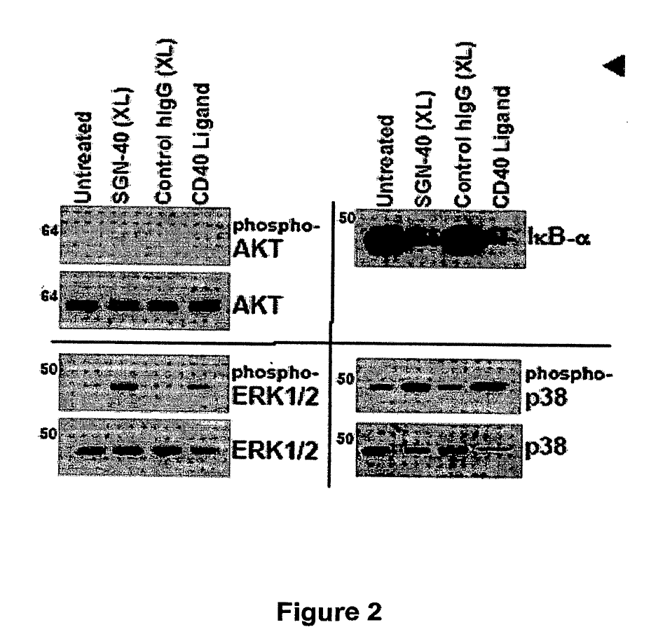

Humanized anti-CD40 antibody (Hu sgn-0; also referred to as SGN-40) binding to CD40⁺ NHL cells activates signaling through the ERK1/2 MAP Kinase, p38 MAP Kinase, and NFkB pathways. Ramos cells (cultured in 2% serum) were stimulated with the humanized CD40 antibody cross-linked (XL) with anti-human IgG for 15 min. Activation of signaling was detected by western blot analysis with antibodies recognizing phosphorylation of ERK1/2 (Thr202/Tyr204), p38 (ThrlSO/Tyr182), and AKT (Ser473). NFkB activation was detected by measuring the degradation of IkB-a protein. Referring to FIG. 2, SGN-40 activated the stress-induced p38 MAP kinase and pro-survival pathways including NF-kB, p42/44 MAP kinase. AKT signaling was modestly elevated by the humanized CD40 antibody.

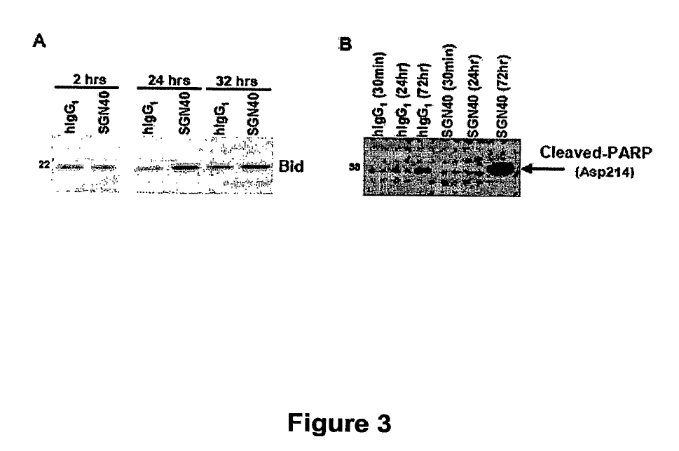

In a further study, Ramos cells (2% FBS) were treated with cross-linked humanized CD40 antibody or control hIgG over a 72 hr time course (1.0 mg/ml mAb). Normalized protein extracts were analyzed by western blotting analysis for the pro-apoptotic Bcl-2 family member Bid. Referring to FIG. 3A, cross-linked anti-CD40 antibody upregulated Bid. Referring to FIG. 3B, degradation of the caspase-3/7 substrate poly(ADP-ribose) polymerase (cleaved-PARP) was monitored over time by western blotting with an antibody recognizing PARP cleaved at Asp214. Consistent with the apoptosis-inducing activity of the humanized CD40 antibody, cleavage of caspase-3 and its downstream substrate poly (ADP-ribose) polymerase was detected in NHL cell lines. Antibody-mediated signaling was qualitatively similar to that mediated by trimeric recombinant humanized CD40 ligand (rhCD40L) (data not shown). However, the overall magnitude of signaling was lower with the humanized CD40 antibody compared to rhCD40L, consistent with the partial agonistic properties of SGN-40.

In addition, constitutive phospho-AKT levels, a key pro-survival signal, were found to be very low in most high-grade lymphoma cell lines and primary NHL specimens, in contrast to the high levels reported in carcinomas. Low AKT activity may bias lymphoma cells toward apoptosis in response to SGN-40 signaling.

### Example 3

### In Vitro Studies with Drug Combinations

A humanized CD40 antibody (SGN-40) enhances the activity of several chemotherapeutic agents against Ramos NHL cells. Two-fold serial dilutions of chemotherapeutic drugs were added to Ramos cells (cultured in 2% FBS) with or without SGN-40 (30.0 ng/ml-0.0586 ng/ml) crosslinked by F(ab′)₂ fragments of a goat antibody specific for the Fcγ region of human IgG. Cells were treated for 72 hours, then labeled with ³H-thymidine for 4 hours to measure proliferation rates. Dose response curves of drugs alone and in combination with the humanized CD40 antibody were reduced in Excel, and Combination Indices (CI) determined using the Calcusyn analysis package (Biosoft). CI values significantly lower than 1.0 indicate synergism. CI values significantly greater than 1.0 indicate antagonism. CI values equal to 1.0 indicate an additive integration. For these studies, n=3 unless otherwise indicated.

Referring to the follow Table 4, the humanized CD40 antibody has additive activity when combined with cisplatin, melphalan, or mitoxantrone and is synergistic with bleomycin.

TABLE 4
In Vitro Drug Combinations

| Drug | ED₅₀ | ED₇₅ | ED₉₀ | Effect |
|---|---|---|---|---|
| Ara-C | 0.95 | 0.90 | 0.90 | Additive |
| Bleomycin | 0.78 | 0.57 | 0.48 | Synergistic |
| Cisplatin | 1.04 | 0.89 | 0.78 | Additive → Syn. |
| Melphalan | 0.91 | 0.90 | 0.96 | Additive |
| Mitoxantrone | 0.70 | 0.97 | 0.99 | Syn. → Add. |
| (n = 2) | | | | |
| TNFα | 0.70 | 0.61 | 0.60 | Synergistic |
| (n = 2) | | | | |
| Etoposide | 1.78 | 1.18 | 0.97 | Antagonistic. → Add. |

### Example 4

### Anti-Tumor Activity of Humanized CD40 Antibody

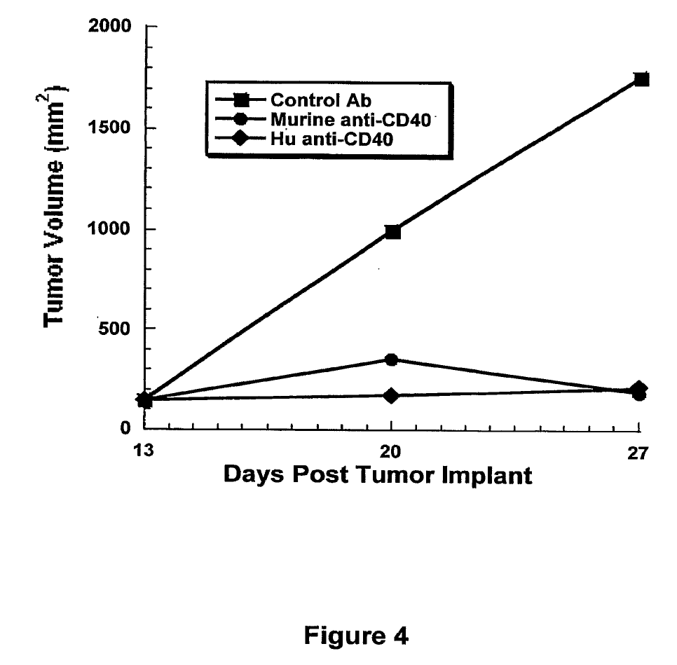

The anti-tumor activity of the humanized CD40 antibody was assayed in a SCID mouse lymphoma xenograft model. Five million Ramos tumor cells were injected subcutaneously into SCID mice (10/group) thirteen days prior to starting drug treatment. Murine CD40 antibody or the humanized S2C6 was given intra-peritoneally 3 times per week (4 mg/kg/dose) with 8 or 5 doses administered. Mice were examined for tumor growth, and tumor volume was measured weekly during the 14-day study period. The results in FIG. 4 show a nearly 9-fold increase in the growth of tumors in control mice, whereas over the same time period, tumor growth in mice treated with either murine CD40 antibody or humanized S2C6 was negligible. The data demonstrate that the humanized antibody was as effective as the murine CD40 antibody in suppressing tumor growth in this B lymphoma xenograft model.

### Example 5

### Prolonged Survival by Humanized CD40 Antibody

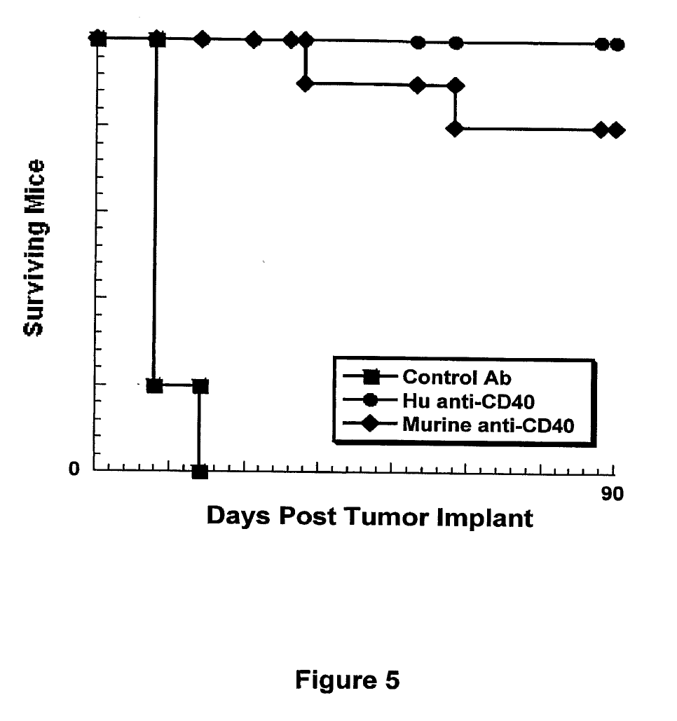

The efficacy of the humanized CD40 antibody on survival of tumor-bearing mice was assayed in a SCID mouse lymphoma xenograft model. SCID mice (10/group) were inoculated intravenously with one million Ramos tumor cells three days prior to antibody treatment. Mice were treated with murine or humanized CD40 antibody, or an Ig control, administered intraperitoneally two times per week (4 mg/kg/dose) for a total of five doses. The mouse cages were examined daily for mortality. The results shown in FIG. 5 show that none of the mice treated with a control antibody survived beyond day 34 post tumor inoculation, whereas eight of the ten mice treated with murine CD40 antibody and all ten mice treated with the humanized CD40 antibody remained alive at even 90 days after tumor implant. The data demonstrate that the humanized antibody was as effective as the murine CD40 antibody in prolonging survival of SCID mice in this B lymphoma xenograft model.

### Example 6

### Combination Therapy

To model localized lymphoma, 5×10⁶ Ramos Burkitt's lymphoma cells were implanted subcutaneously into the right flank of C.B.-17 SCID mice (Harlan, Indianapolis, Ind.). Therapy was initiated when the mean tumor size in each group of five animals was 100 mm³. CHOP was used for a single course, alone or in combination with humanized CD40 antibody (hu sgn-0; SGN-40), using the following dose schedule: cyclophosphamide at 30 mg/kg, i.v. qld x 1; doxorubicin at 2.475 mg/kg, i.v. qld x 1; vincristine at 0.375 mg/kg, i.v. qld x 1; and prednisone at 0.15 mg/kg, p.o. q1d x 5. The CD40 antibody was administered i.p. at 4 mg/kg q4d x 4 when used alone or in combination with prednisone or CHOP. Tumor size was determined using the formula (L×W²)/2.

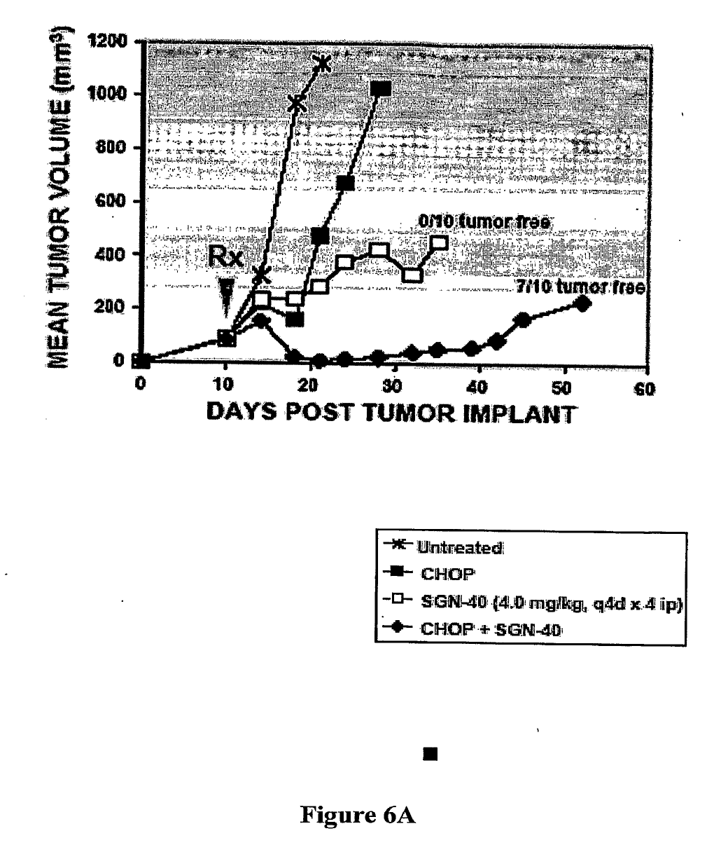

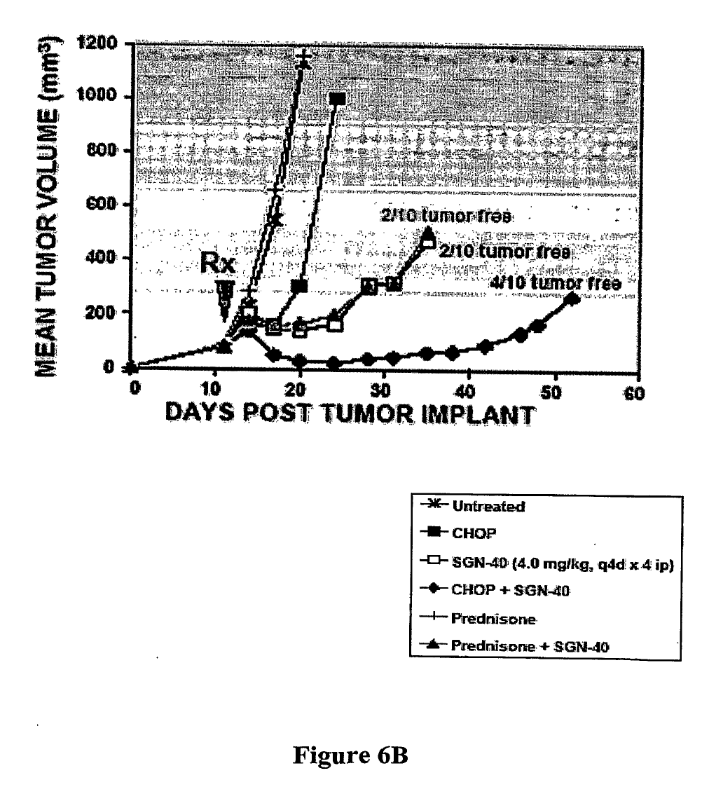

Referring to FIG. 6A, the initiation of therapy is indicated by Rx. Treatment with CHOP, prednisone, CD40 antibody, prednisone with CD40 antibody and CHOP with CD40 antibody was compared with that of the untreated control. Treatment with CHOP alone has little effect on tumor growth. Treatment with CHOP and humanized CD40 antibody was substantially better than the other treatments.

To compare the effect of SGN-40 treatment alone or in combination with CHOP or prednisone, SCID mice were subcutaneously implanted with Ramos cells, and dosed with SGN-40 alone or in combination with CHOP as described above. In addition, prednisone steroid was administered alone (0.15 mg/kg, qld x 5 po) or in combination with SGN-40 (4.0 mg/kg, q4d x 4 ip).

Referring to FIG. 6B, CHOP or prednisone had little effect when administered alone. In contrast, both CHOP and prednisone showed greater efficacy when administered in combination with SGN-40.

In an additional study, the humanized CD40 antibody SGN-40 was shown to be similarly effective in promoting survival in a Xenograft model. Briefly IM-9 cells (1×10⁶ per mouse) were introduced intravenously into SCID mice in a disseminated xenograft model. Animals were treated 3 days post tumor cell injection with either SGN-40(4.0 mg/kg, q2d x9, ip) or prednisone (0.15 mg/kg, qld x5, po) alone, or in combination. Survival data was plotted using a Kaplan-Meier curve (data not shown). As stated above, the efficacy of SGN-40 was essentially the same in the presence or absence of prednisone.

### Example 7

### SGN-40 and Rituximab have Comparable Activity In a NHL Xenograft Model

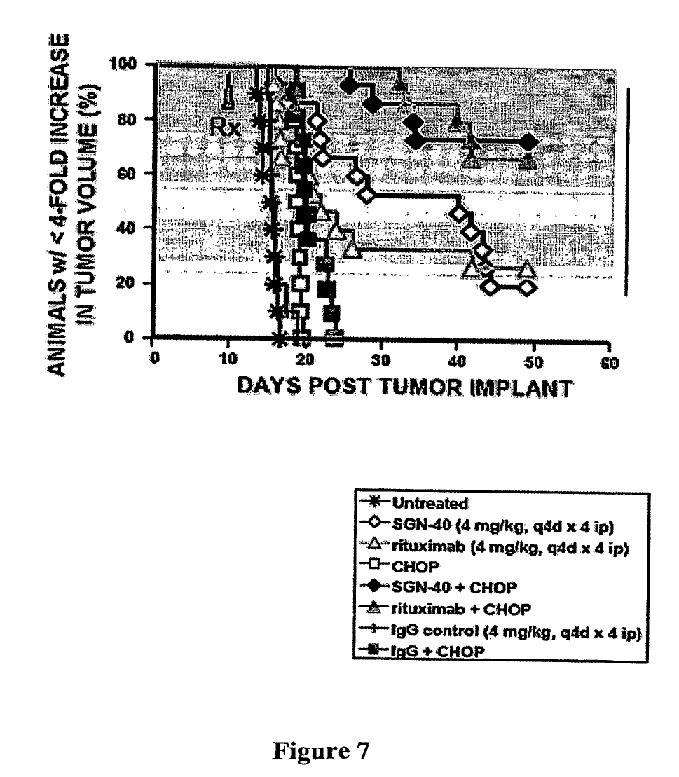

To compare the efficacy of SGN-40 and Rituximab, SCID mice were subcutaneously implanted with 5×10⁶ Ramos cells, and tumors grown to 100 mm³ prior to treatment. Ramos tumor growth data was plotted aspercent of mice in each group with <4-fold increase in tumor volume using a Kaplan-Meier plot (GrapbPad Prism). Mice were dosed with SGN-40 or Rituximab individually (4 mg/kg, q4d x 4 ip) or in combination with CHOP chemotherapy (CHO, qld x 1, iv; P, qld x 5, po). Referring to FIG. 7, SGN-40 and Rituximab exhibited similar efficacy alone or in combination with CHOP.

### Example 7

### Escalated Dosing Schedule

An open-label, multi-dose, single arm, Phase I protocol initiated at five clinical sites. Initially, enrolled patients, diagnosed with multiple myeloma, received weekly intravenous infusions of a humanized CD40 antibody (hu sgn-0) with a cohort-specific dose level of 0.5, 1, 2 and 4 mg/kg/wk. Because of Grade 3 cytokine release syndrome associated with the first dose at the 4 mg/kg level, the study was amended to allow intra-patient dose escalation over two weeks. Cohort specific doses of 3, 4, 6 and 8 mg/kg/week were used. The patient eligibility criteria included: patients must have failed at least two prior systemic therapies; at least four weeks since chemotherapy; patients who experience a minimal response (MR) or greater are eligible for a second cycle of therapy.

The revised dose escalation schema are shown in the following Table 5.

TABLE 5

| | Week 1 | | Week 2 | Week 3 | Week 4 | Week 5 |
|---|---|---|---|---|---|---|
| Cohort | Day 1 | Day 4 | Day 8 | Day 15 | Day 22 | Day 29 |
| I | 1 | 1 | 2 | 3 | 3 | 3 |
| II | 1 | 1 | 2 | 4 | 4 | 4 |
| III | 1 | 1 | 2 | 4 | 6 | 6 |
| IV | 1 | 1 | 2 | 4 | 6 | 8 |

The patient demographics were as follows: Gender: Male (14), Female (9); Median age: 61 years (range 40-77); Race: Caucasian (17), Black (2), Hispanic (2), Other (2); Median time from initial diagnosis: 6.3 years (range 1.7-14.1); Median number of prior therapies: 5.5 (range 2-10), including Bortezomib, 9; Thalidomide 15; Revlimid™, 4; Melphalan, 6; Combination chemo, 17; and Auto transplant, 5.

Patients were monitored for adverse events. Administration of the humanized CD40 antibody appeared to trigger cytokine release, and TNF-alpha levels in the plasma are elevated following the first infusion only. The drug-loading period reduced the first-dose cytokine release syndrome. Following amendment of the protocol, symptoms of first dose-associated cytokine release was markedly reduced eliminated. Further, even at the low doses tested thus far, there is preliminary evidence for antitumor activity

### Example 8

### Pharmacokinetics

The humanized CD40 antibody, SGN-40, was administered twice during the first week and once a week 40 was administered twice during the first week and once a week for the next 4 weeks to patients with multiple myeloma. Patients who did not receive the final dose were excluded from analysis. The peak serum concentration (Cₘₐₓ) of SGN-40 observed following the last dose on Day 29 and the subsequent trough levels 7 days later on Day 36 are shown for each cohort in Table 6. The half-life after the last infusion and the area under the concentration time curve from Days 0 to 50 from a 1 compartment model fit of the data were also calculated. (n/a means not applicable.) Data are presented as the mean±standard deviation also shown. Data are presented as the mean±standard deviation (N>3) with the exception of Cohort I Day 36 Cmin (N=2), and Cohort II, T1/2 and AUC₀₋₅₀ (N-1). The patients maintained a plasma SGN-40 concentration of at least about 10 micrograms/ml.

TABLE 7

| | Day 29 | | | AUC₀₋₅₀ |
|---|---|---|---|---|
| Cohort | Cₘₐₓ (μg/mL) | Day 36 Cₘᵢₙ (μg/mL) | T ½ (Days) | (μg-d/mL) |
| I | 45.40 ± 14.31 | 9.94 ± n/a | 2.67 ± 1.08 | 653.93 ± 154.65 |
| II | 143.00 ± 27.78 | 69.93 ± 14.08 | 4.61 ± n/a | 2086.02 ± n/a |
| III | 149.25 ± 29.34 | 41.57 ± 19.22 | 3.85 ± 1.37 | 1988.88 ± 439.74 |
| IV | 244.25 ± 96.00 | 112.84 ± 50.40 | 5.39 ± 1.84 | 3441.73 ± 1382.79 |

Various references, including patent applications, patents, and scientific publications, are cited herein, the disclosures of which are incorporated herein by reference in their entireties. Citation or identification of any reference herein shall not be construed as an admission that such reference is available as prior art to the present invention.

The application of the teachings disclosed herein is not to be limited in scope by the specific embodiments described herein. Indeed, various modifications will be within the capabilities of one having ordinary skill in the art in light of the teachings contained herein and accompanying examples. Such modifications are intended to fall within the scope of the appended claims.

1. A method for the treatment or prevention of a CD40-associated disorder, comprising:
(a) administering to a patient in need thereof an initial dose of a CD40 binding agent which (i) immunospecifically binds to CD40; and (ii) increases the binding of CD40 ligand to cell surface CD40 on B cells by at least 45% (b); and
(b) administering to the patient a second dose of the CD40 binding agent;
wherein the initial dose is less than the second dose, whereby the patient exhibits reduced cytokine release;
wherein the CD40 binding agent inhibits the growth or differentiation of cells of the CD40-associated disorder.

2. The method of claim 1, wherein the initial dose is about 0.5 mg/kg, about 1 mg/kg, about 2 mg/kg, about 3 mg/kg or about 4 mg/kg.

3. The method of claim 2, wherein the second dose is about 1 mg/kg to about 16 mg/kg.

4. The method of claim 3, wherein the second dose is about 1 mg/kg to about 8 mg/kg.

5. The method of claim 1, wherein the initial dose and the subsequent dose are administered on consecutive days.

6. The method of claim 1, wherein the subsequent dose is administered two, three, four or seven days after the initial dose.

7. The method of claim 1, further comprising administering a third dose of the CD40 binding agent to the patient, wherein the third dose is greater than or equal to the second dose.

8. The method of claim 1 or 7, whereby an adverse event associated with administration of the CD40 binding agent is reduced.

9. The method of claim 1, further comprising administering two initial doses to the patient prior to administration of the second dose, wherein the initial doses are the same.

10. The method of claim 9, wherein the initial doses are administered two, three or four days apart.

11. The method of claim 1, further comprising: administering to the patient a therapeutic agent, wherein the therapeutic agent reduces cytokine release induced by the CD40 binding agent.

12. The method of claim 12, wherein the therapeutic agent is a steroid or an immunomodulatory agent.

13. The method of claim 1, wherein the CD40 binding agent is a humanized, chimeric or human antibody.

14. The method of claim 1, wherein the CD40 binding agent comprises a humanized heavy chain variable domain comprising a framework region having an amino acid sequence at least 90% identical to the amino acid sequence of the framework region of the human variable domain heavy chain subgroup III consensus amino acid sequence of SEQ ID NO:2, and comprising at least one CDR having an amino acid sequence at least 90% identical to a corresponding heavy chain CDR of SEQ ID NO:3.

15. The method of claim 1, wherein the CD40 binding agent comprises a humanized light chain variable domain comprising a framework region having an amino acid sequence at least 90% identical to the framework region of the human variable domain light chain subgroup kappa I consensus amino acid sequence of SEQ ID NO:13, and comprising at least one CDR having an amino acid sequence at least 90% identical to a corresponding light chain CDR of SEQ ID NO:14.

16. The method of claim 14, wherein the CD40 binding agent further comprises a humanized light chain variable domain comprising a framework region comprising an amino acid sequence at least 90% identical to the framework region of the human variable domain light chain subgroup kappa I consensus amino acid sequence of SEQ ID NO:13 comprising at least one CDR having an amino acid sequence at least 90% identical to a corresponding light chain CDR of SEQ ID NO: 14.

17. The method of claim 14, wherein each heavy chain CDR is at least 90% identical to the corresponding heavy chain CDRs of SEQ ID NO:3.

18. The method of claim 1744, wherein the heavy chain CDRs comprise the amino acid sequences of the corresponding heavy chain CDRs of SEQ ID NO:3.

19. The method of claim 15 or 16, wherein each light chain CDR is at least 90% identical to the corresponding light chain CDR of SEQ ID NO:14.

20. The method of claim 19, wherein the light chain CDRs comprise the amino acid sequences of the light chain CDRs of SEQ ID NO:14.

21. The method of claim 14, wherein the heavy chain variable domain comprises the amino acid sequence of SEQ ID NO:3, SEQ ID NO:4, SEQ ID NO:5, SEQ ID NO:6, SEQ ID NO:7, SEQ ID NO:8, SEQ ID NO:9, SEQ ID NO:10, or SEQ ID NO:11.

22. The method of claim 15, wherein the light chain variable domain comprises the amino acid sequence of SEQ ID NO:14, SEQ ID NO:15, or SEQ ID NO:16.

23. The method of claim 16, wherein the heavy chain variable domain comprises the amino acid sequence of SEQ ID NO:3, SEQ ID NO:4, SEQ ID NO:5, SEQ ID NO:6, SEQ ID NO:7, SEQ ID NO:8, SEQ ID NO:9, SEQ ID NO:10, or SEQ ID NO:11, and the light chain variable domain amino acid sequence of SEQ ID NO:14, SEQ ID NO:15, or SEQ ID NO:16.

24. The method of claim 13, wherein the CD40 binding agent further comprises a human IgG constant region.

25. (canceled)

26. The method of claim 24, wherein the isotype of the IgG constant region is IgG1.

27. The method of claim 15 or 6, wherein the light chain constant domain is a kappa constant domain.

28-32. (canceled)

33. The method of claim 13, wherein the antibody is hu sgn-0, hu sgn-1, hu sgn-2, hu sgn-4, hu sgn-14, hu sgn-15, hu sgn-16, hu sgn-17, hu sgn-18, hu sgn-19, hu sgn-22, hu sgn-23, hu sgn-26 or hu sgn-27.

34. The method of claim 1, wherein the CD40 binding agent is an antigen-binding antibody fragment.

35. (canceled)

36. The method of claim 1, wherein the CD40-associated disorder is chronic lymphocytic leukemia, multiple myeloma, non-Hodgkin's lymphoma, Hodgkin's disease or Waldenstrom's macroglobulinemia.

37. The method of claim 36, wherein the CD40-associated disorder is non-Hodgkin's lymphoma.

38. The method of claim 37, wherein the non-Hodgkin's lymphoma is diffuse large B cell lymphoma.

39. The method of claim 1, wherein the CD40-associated disorder is an autoimmune disease.

40. The method of claim 1, further comprising: administering to a patient in need thereof (c) a therapeutic agent; wherein the therapeutic agent exerts a cytotoxic or cytostatic effect on the CD40 expressing cell.

41. The method of claim 40, wherein the therapeutic agent is not a CD20 antibody, cycloheximide or thalidomide.

42. The method of claim 40, wherein the therapeutic agent inhibits the Akt survival pathway.

43. The method of claim 40, wherein the therapeutic agent reduces or inhibits phospho-AKT levels.

44. The method of claim 40, wherein the therapeutic agent is bortezomib, bleomycin, lenalidomide, gemcitabine, CHOP, R-CHOP, ICE or R-ICE.

45. The method of claim 44, wherein the therapeutic agent is CHOP, and wherein Rituximab is not administered to the patient.

46. The method of claim 40, wherein the CD40 binding agent and the therapeutic agent exhibit a synergistic effect.

47. The method of claim 40, wherein the CD40 binding agent and the therapeutic agent exhibit an additive effect.

48. The method of claim 2, wherein the initial dose is at least 4 mg/kg and a therapeutic agent is administered to the patient, wherein the therapeutic agent reduces cytokine release induced by the CD40 binding agent.

49. The method of claim 48, wherein the therapeutic agent is administered prior to administration of the CD40 binding agent.

50. The method of claim 1, wherein the patient receives at least one cycle of at least four doses of the CD40 binding agent.

51. The method of claim 1, wherein the patient receive at least one cycle of at least five doses of the CD40 binding agent.

52. The method of claim 50 or 51, wherein the patient receives at least two cycles of the CD40 binding agent.

53. The method of claim 50, wherein the patient maintains a blood plasma level of at least 5 μg/ml after the initial dose and during the cycle.

54. The method of claim 53, wherein the patient maintains a blood plasma level of at least 10 μg/ml after the initial dose and during the cycle.
